SEQUENCE LISTING (2 sequences)

SEQ ID NO: 1 - DNA, 20 bp, Artificial Sequence
  Synthetic
gggtgggaaaatagaccaat

SEQ ID NO: 2 - DNA, 20 bp, Artificial Sequence
  Synthetic
taggaaggggataagtaaca

(19) United States
(12) Patent Application Publication
(10) Pub. No.: US 2022/0280571 A1
(43) Pub. Date: Sep. 8, 2022

(54) COMPOSITIONS AND METHODS FOR TREATING ALPHA THALASSEMIA

(71) Applicants: THE REGENTS OF THE UNIVERSITY OF CALIFORNIA, Oakland, CA (US); The J. David Gladstone Institutes, a testamentary trust established under the Will of J. David Glads, San Francisco, CA (US); THE BOARD OF TRUSTEES OF THE LELAND STANFORD JUNIOR UNIVERSITY, Stanford, CA (US)

(72) Inventors: Tippi MacKenzie, San Francisco, CA (US); Beeke Wienert, San Francisco, CA (US); Michael Kyle Cromer, Churchville, VI (US); Matthew H. Porteus, Portola Valley, CA (US)

(21) Appl. No.: 17/631,240

(22) PCT Filed: Jul. 31, 2020
(86) PCT No.: PCT/US20/44562

§ 371 Date: Jan. 28, 2022

Related U.S. Application Data
(60) Provisional application No. 62/881,726, filed on Aug. 1, 2019.

(51) Int. Cl. A61K 35/28; A61K 48/00; A61K 38/42; A61K 38/46; A61K 31/7088; A61P 7/06

(52) U.S. Cl. CPC A61K 35/28; A61K 48/0075; A61K 48/0008; A61K 38/42; A61K 38/465; A61K 31/7088; A61P 7/06

(57) ABSTRACT

The disclosure provide methods and compositions that use gene editing or gene therapy to treat alpha thalassemia major. The gene editing may be performed ex vivo in fetal cells or cells obtained after birth to improve production of globin, with those cells then delivered to the fetus. In other embodiments, gene editing reagents are delivered to the fetus or the patient after birth in vivo to edit genes of the alpha-globin cluster and improve globin production. Gene editing system such as CRISPR, TALENs, or ZFNs are used to increase production of alpha, zeta, or theta globin and/or to decrease production of gamma globin. Globin production may be improved by inserting a copy of globin gene or mutating a globin gene to change its expression. Any of the gene editing strategies may be performed in conjunction with delivering to a fetus or patient after birth a therapeutic blood transfusion. Exemplary patients after birth are patients no older than one year of age.

### CROSS-REFERENCE TO RELATED APPLICATIONS

This application claims the priority benefit under 35 U.S.C. § 119(e) of U.S. Provisional Patent Application No. 62/881,726, filed Aug. 1, 2019, which is incorporated herein by reference in its entirety.

### TECHNICAL FIELD

The invention relates to treatment of alpha thalassemia using gene editing tools.

### BACKGROUND

Alpha thalassemia major (ATM) is a blood disorder affecting babies in the womb. ATM is usually only detected in the first or second trimester of pregnancy and is almost always fatal unless blood transfusions are performed before birth. Fetuses with homozygous alpha thalassemia usually die in the second trimester of pregnancy or soon after birth. Alpha thalassemia is a hereditary disorder caused by deficient or absent production of alpha-globin. Alpha-globin gene mutation frequency is high among many populations, and the severe form has the highest prevalence in Southeast Asia. Even patients with two or three gene deletions/mutations can have symptomatic anemia and require transfusions after birth; these patients would also benefit from the strategies described and methods disclosed herein.

Alpha thalassemia major (ATM) is an autosomal recessive condition resulting from inheritance of mutations in all four alpha-globin genes (two on each chromosome). ATM can be lethal in utero, necessitating fetal therapy such as using blood transfusions. Blood transfusions may help a fetus or infant survive, but do not cure the underlying condition. In fact, there is no known cure for alpha thalassemia, including alpha thalassemia major, highlighting the need in the art for effective therapies to treat this disease.

### SUMMARY

The disclosure provides methods and compositions for treating patients such as babies, in the womb or postnatally, who have alpha thalassemia major (ATM). Disclosed herein is the surprising realization that gene therapy can be administered to fetuses and newborns, e.g., patients no older than one year of age such as patients no older than one year of age, e.g., patients no older than three months of age, in addition to older individuals including adults. The treatment uses gene editing or gene therapy to manipulate globin expression to treat ATM. The gene editing may be performed ex vivo in fetal or adult cells to improve production of globin, with those cells then delivered to the fetus or to the subject after birth. In some embodiments, gene editing reagents are delivered into fetal circulation in vivo to edit genes of the alpha-globin or beta globin cluster and improve globin production in the fetus. Specifically, gene editing systems such as CRISPR, TALENs, or ZFNs are used to increase production of alpha, zeta, or theta globin and/or to decrease production of gamma globin or beta globin. Other embodiments involve gene therapy to deliver and express a globin gene using, e.g., a lentiviral vector. The viral vector can contain the alpha-globin or zeta-globin coding sequence with or without non-coding introns and regulatory regions such as a promoter or enhancer. The introns and regulatory regions may also be derived from beta-globin and include the beta-globin promoter, full-length or truncated introns and enhancer elements from the locus control region. Globin production may be improved by providing a copy of a globin gene or mutating a globin gene or its regulatory region to change its expression. Any of the gene editing strategies of the disclosure may beneficially be performed in conjunction with delivering to a fetus a therapeutic blood transfusion. The gene therapy or editing may be done before or after birth. Before or after birth, the gene therapy or editing may be done in vivo, by injecting the reagents directly into the circulation, or ex vivo, by taking HSCs from the fetus, the cord blood, or the peripheral blood or bone marrow of the patient, e.g., after birth.

In certain aspects, the disclosure provides methods of treating alpha thalassemia. Methods may include introducing, into a fetal or adult cell or into circulation in a subject (fetal or postnatal), a globin gene and gene editing reagents, whereby the gene editing agents cause insertion of the globin gene into genomic material and cause the globin gene to be expressed.

In ex vivo embodiments, the methods may include obtaining cells such as hematopoietic stem cells (HSCs), red blood cells (RBCs), or precursors thereof, from the subject, using the gene editing reagents to introduce the globin gene, and introducing the cells or their progeny into circulation in the subject by injection. The injection may be into an umbilical vein, placenta, or fetal liver or heart.

For in vivo embodiments, the methods may include surgically accessing the fetus in vivo in a pregnant subject and injecting the reagents into the fetal circulation (by injecting into the umbilical vein or fetal heart, fetal liver, or the placenta) in vivo, in a manner compatible with maintenance of the pregnancy, as would be known in the art.

Any suitable gene editing reagents may be used. For example, the gene editing reagents may include at least one Cas endonuclease or a nucleic acid encoding the Cas endonuclease. In some embodiments, the globin gene is an alpha-globin gene and the gene editing reagents include one or more guide RNAs that target delivery of the alpha-globin gene to a predetermined locus in the genomic material. The target locus may be, for example, an alpha-globin gene cluster in chromosome 16, a beta globin gene cluster in chromosome 11, or a genomic safe harbor locus, such as an AAVS1, cCR5, CLYBL, or hROSA26 genomic safe harbor. Genomic safe harbors are sites in the genome able to accommodate the integration of new genetic material in a manner that ensures that the newly inserted genetic elements function predictably and do not cause alterations of the host genome that pose a risk to the host cell or organism. Methods may include performing the introducing step while delivering to the fetus a therapeutic blood transfusion with blood that includes alpha-globin-expressing red blood cells.

In DNA sense embodiments, the nucleic acid encoding the Cas endonuclease and the globin gene may be packaged using one or more lentiviral or adeno-associated virus (AAV) vector. In some embodiments, the globin gene is included as a segment of DNA that also includes one or more of a promoter, a fluorescent protein as an expression marker, an SV40 sequence, and a poly(A) sequence. In mRNA-sense embodiments, the globin gene may be included as DNA and the gene editing reagents may include at least one mRNA that, when is introduced into the fetus or HSC, is translated into a gene editing nuclease by a ribosome. In protein embodiments, the gene editing reagents may include at least a first Cas ribonucleoprotein, such as a Cas9 ribonucleoprotein (RNP), that includes a first guide RNA (gRNA) that binds the RNP to a locus within a globin gene cluster in the genomic material and introduces the globin gene into the locus within the globin gene cluster.

Aspects of the disclosure provide a composition for treatment of alpha thalassemia. The composition includes a globin gene and gene editing reagents that, when the composition is introduced into a subject, as a fetus or in a postnatal period, insert the globin gene into genomic material. The subject may be a fetus or a patient after birth, such as a patient no older than one, two, or three months of age. Of course, the therapeutics and therapeutic methods disclosed herein are suitable for use in patients of age, such as patients no older than one year of age, as well. In certain embodiments, the globin gene is an alpha-globin gene and the gene editing reagents comprise a first Cas9 ribonucleoprotein (RNP) that includes a first guide RNA (gRNA) and a second Cas9 RNP, wherein the first Cas9 RNP and the second Cas9 RNP bind to a locus within an alpha-globin gene cluster in chromosome 16 of the genomic material, and introduce the coding sequence of an alpha-globin gene with or without non-coding introns and regulatory sequences into the locus within the alpha-globin gene cluster. The introns and regulatory sequences can be derived from an alpha-globin gene or from a beta-globin gene.

In DNA sense embodiments, the gene editing reagents are included as DNA in a vector that is transcribed after the composition is introduced into the fetus or into fetal cells such as fetal HSCs, or that is introduced into the cells of a patient after birth, such as the HSCs of a newborn.

The gene editing reagents may include transcription activator like effector nucleases (TALENs) or zinc-finger nucleases (ZFNs), or nucleic acid encoding the TALENs or the ZFNs, in which the TALENs or the ZFNs are designed to introduce the alpha-globin gene into a locus within an alpha-globin gene cluster in chromosome 16.

Preferably, the gene editing reagents are targeted to a predetermined locus in the genomic material such as an alpha-globin gene cluster in chromosome 16, a beta globin gene cluster in chromosome 11, or a genomic safe harbor. The globin gene may be included as DNA and the gene editing reagents may include at least one mRNA that, when the composition is introduced into a fetus or fetal cells, or the cells of a patient after birth, e.g., newborn, is translated into a gene editing nuclease by a fetal ribosome.

The composition may include one or more viral delivery vectors (such as a lentivirus or an adeno-associated virus) containing the globin gene (e.g., alpha- or zeta-globin gene) or the gene editing reagents. The delivery vectors may further comprise at least one regulatory region and/or intron and/or poly A tail from a globin gene, such as a beta-globin gene. The delivery vector may include a surface modification that targets the vector to a cell of the subject, such as an antibody linked to an external surface of the viral delivery vector, wherein the antibody targets hematopoietic stem cells, or precursors thereof. The composition may include a particle (e.g., lipid nanoparticle or liposome) containing the globin gene and the gene editing reagents, or a plurality of lipid nanoparticles having the globin gene and the gene editing reagents comprised or embedded therein. For example, the plurality of lipid nanoparticles may include at least: a first solid lipid nanoparticle comprising a segment of DNA that includes the globin gene; a second solid lipid nanoparticle that includes at least one Cas endonuclease complexed with a guide RNA (gRNA) that targets the Cas endonuclease to a locus within an alpha-globin gene cluster in chromosome 16. The particle(s) may be provided as one or a plurality of liposomes enveloping one or more of the globin gene and the gene editing reagents.

In certain embodiments, the composition for treatment of alpha thalassemia comprises a zeta-globin gene as a replacement or substitute gene for a malfunctioning alpha-globin gene in an ATM subject. The zeta-globin gene (e.g., that is provided for gene replacement) may include a mutation in a repressor region of the gene or gene regulatory element in proximity to the gene. The repressor region may include a BCL11A binding site, ZBTB7A binding site, a RREB1 binding site, or NF-kB binding region in a 5′ or 3′ portion of the zeta-globin gene. The zeta-globin gene may include a mutation in a 3′ sequence of a transcribed region such that—when the composition is introduced into a fetal HSC—the zeta-globin gene is transcribed into zeta-globin transcripts that are more stable than transcripts from a wild-type zeta-globin gene that is the same as the zeta-globin gene but that does not have the mutation in the 3′ sequence of the transcribed region. In some embodiments, after the composition is injected into fetal circulation, the fetus expresses the globin gene for at least a trimester.

In certain embodiments, the composition is provided as a kit. The kit may include additional reagents that promote integration of the globin gene into the genomic material, wherein the additional reagents include one or more of a polymerase, a ligase, dNTPs, a co-factor, and a topoisomerase. The kit may include one or more surgical tools for delivering the globin gene and the gene editing reagents into a fetal or newborn (less than one year of age) circulation, or the circulation of a patient older than one year of age. The kit may further include a blood bag with blood comprising alpha globin for transfusion into the fetus.

The composition may be used in a method of treating alpha thalassemia. The method includes obtaining a sample comprising cells (such as HSCs, RBCs, or precursors thereof) from a fetus or from cord blood, using the composition to introduce the globin gene into the cells, and introducing the modified cells into the fetus, for example using surgical techniques such as injection into the umbilical vein, fetal liver, fetal heart or placenta. In addition, the method can include a step of validating that the cells and/or their progeny express the introduced globin gene. In like manner, the composition is useful in a method of treating alpha thalassemia in a patient after birth, e.g., newborn (no older than one year of age), comprising obtaining a cell sample from such an individual (e.g., HSCs, RBCs or precursors thereof), introducing the composition into the cells of the patient after birth, e.g., newborn, and introducing the modified cells into the individual, e.g., using surgical techniques such as injection. As with the modification of fetal cells, the modification of cells of a patient after birth, e.g., newborn, can be validated to ensure that the cells and/or their progeny express the introduced globin gene.

Other aspects of the disclosure provide a method for treating alpha thalassemia by modulating the expression of one or more globin genes. The method includes introducing, into a cell, e.g., fetal cell, or fetus, gene editing reagents that (i) increase production of alpha, zeta, or theta globin, or (ii) decrease production of gamma globin. In ex vivo embodiments, the cell is an HSC or RBC (or precursor thereof) from the fetus. In in vivo embodiments, the gene editing reagents are injected into circulation in the fetus during a surgical procedure. The gene editing reagents may include a Cas endonuclease; a nucleic acid encoding the Cas endonuclease; a transcription activator-like effector nuclease (TALEN); a nucleic acid encoding the TALEN; a zinc-finger nuclease (ZFN); or a nucleic acid encoding the ZFN. Analogous methods are also contemplated for the treatment of postnatal ATM subjects.

In some embodiments, the gene editing reagents introduce a change into a sequence within a zeta-globin gene (e.g., a mutation into a repressor region such as a BCL11A, ZBTB7A or NF-kB binding region in a 5′ or 3′ end of the zeta-globin gene; or a mutation into a 3′ sequence of a transcribed region of the zeta-globin gene) that, for example, stabilizes zeta-globin transcripts.

The method may include performing the introducing step while delivering to the fetus or patient after birth, e.g., newborn, a therapeutic blood transfusion with blood that includes alpha globin. The gene editing reagents may be provided at least in part in a viral vector or non-viral particle for delivery. The gene editing reagents may further introduce a promoter or a transcription factor binding site to increase, or control, transcription of the zeta-globin gene. The method, e.g., as performed on fetuses, may further include inhibiting gene silencing of a zeta-globin gene and increasing persistence of zeta globin into at least a second trimester by injecting into the fetus via a needle the gene editing reagents, wherein the gene editing reagents include a Cas endonuclease gene and a DNA-sense guide RNA packaged in a viral vector, wherein, when the gene editing reagents are expressed in the fetus, the gene editing reagents introduce a mutation into a repressor region in the zeta-globin gene or introduce a transcription-stabilizing mutation into a 3′ sequence of a translated region of the zeta-globin gene. In some embodiments, the gene editing reagents introduce a mutation that activates the zeta-globin gene.

In some embodiments, the method includes: obtaining the cells from the fetus or patient after birth, such as a newborn no older than one year of age and performing the introducing step ex vivo to introduce the change into the zeta-globin gene in the cells, such that the production of zeta globin in the cells is increased. The method then includes delivering the modified cells into fetal circulation by injection into the umbilical vein, liver, heart, or placenta of the fetus.

Certain aspects of the disclosure provide a composition for treatment of alpha thalassemia. The composition includes gene editing reagents that when introduced into a fetus or into fetal hematopoietic stem cells (HSCs) ex vivo introduce a change into a sequence within a globin gene within genomic material of the fetus or the HSC, wherein the change activates or derepresses the globin gene. The gene editing reagents may be provided in a viral vector or non-viral particle that optionally includes one or more targeting molecules that target the viral vector or the non-viral particle to the target cells, such as CD34, CD90, or other molecules found on HSCs.

The gene editing reagents may include a Cas endonuclease, or nucleic acid encoding the Cas endonuclease, and one or more guide RNAs that target the globin gene.

In some embodiments of the composition the globin gene is a zeta-globin gene and the gene editing reagents introduce a mutation into the zeta-globin gene (e.g., one or more mutations into a repressor and or a 3′ sequence of a translated region) such that, after the change is introduced within the sequence of the globin gene within the fetus, the zeta-globin gene is not silenced at the end of a first trimester, and the zeta globin gene is continuously expressed during a second trimester. After the change is introduced within the sequence of the globin gene, the mutated zeta-globin gene is transcribed into modified transcripts, wherein the modified transcripts persist for longer than similar but unmodified transcripts. In alternative embodiments, the mutation activates the zeta-globin gene resulting in persistent zeta-globin expression and activity.

In some embodiments, the composition includes, or is provided in a kit that also includes, normal blood comprising alpha globin for transfusion to the fetus or patient after birth, e.g., newborn no older than one year of age. The gene editing reagents may include CRISPR, TALENS, or ZFNs, and may be included in the composition or the kit in a form that includes DNA, mRNA, or protein. Optionally, the gene editing reagents further introduce a promoter or a transcription factor binding site to increase transcription of the a globin gene of the alpha-globin gene cluster, such as zeta-globin. The kit may also include surgical tools for injection of the composition into fetal or patient after birth (e.g., newborn no older than one year of age) circulation. The kit or the composition may be used in a method of treating alpha thalassemia that includes obtaining a sample comprising cells from a fetal or postnatal subject; using the composition or kit to modify a zeta-globin gene within the cells, and validating that the cells express the modified zeta-globin gene. The cells preferably include HSCs, RBCs, or precursors or progeny thereof, most preferably, fetal HSCs. The method may further include surgically accessing the fetus in the pregnant woman and introducing the modified cells, such as HSCs, into fetal circulation by injecting the modified HSCs into the umbilical vein or the fetal liver, heart, or placenta, whereby the modified zeta-globin gene in the fetus is resistant to gene silencing and is expressed persistently into at least a second trimester.

Thus, consistent with the foregoing disclosure, one aspect of the disclosure provides a composition for treatment of alpha thalassemia in a fetus or patient after birth, such as a patient no older than one year of age, e.g., a patient no older than three months of age, or a cell (e.g., an HSC) thereof, the composition comprising: (a) a globin gene, wherein the globin gene is an alpha-globin gene, a zeta-globin gene, a zeta- or alpha-globin gene associated with at least one beta-globin regulatory region, a zeta- or alpha-globin gene associated with at least one beta-globin intron, or a zeta- or alpha-globin gene associated with at least on beta-globin regulatory region and at least one beta-globin intron; and (b) gene editing reagents that, when the composition is introduced directly into the fetus or patient, or into HSCs derived from the fetus or patient, insert the globin gene into genomic material. In some embodiments, the globin gene is an alpha-globin gene and the gene editing reagents comprise a first Cas9 ribonucleoprotein (RNP) that includes a first guide RNA (gRNA) and a second Cas9 RNP, wherein the first Cas9 RNP and the second Cas9 RNP bind to a locus within an alpha-globin gene cluster in chromosome 16 of the genomic material, and introduce the alpha-globin gene into the locus within the alpha-globin gene cluster. In some embodiments, the gene editing reagents are included as DNA in a vector that is transcribed after the composition is introduced into the fetus or the HSCs. In some embodiments, the vector is a viral vector. In some embodiments, the gene editing reagents comprise transcription activator like effector nucleases (TALENs) or zinc-finger nucleases (ZFNs), or nucleic acid encoding the TALENs or the ZFNs, wherein the TALENs or the ZFNs are designed to introduce the alpha-globin gene into a locus within an alpha-globin gene cluster in chromosome 16. In some embodiments, the gene editing reagents are targeted to a predetermined locus in the genomic material, wherein the locus is selected from: an alpha-globin gene cluster in chromosome 16; an intronic region of the beta-globin gene in chromosome 11, and a genomic safe harbor. In some embodiments, the safe harbor comprises a locus selected from the group consisting of AAVS1, CCR5, CLYBL and hROSA26.

In some embodiments of the composition according to the disclosure, the globin gene is included as a segment of DNA that also includes one or more of a promoter, an intron, a fluorescent protein, an SV40 sequence, and a poly(A) sequence. In some embodiments, the globin gene is included as DNA and the gene editing reagents include at least one mRNA that, when the composition is introduced into a fetus or HSC, is translated into a gene editing nuclease by a fetal or adult ribosome, and a guide RNA for genomic targeting. In some embodiments, the gene editing nuclease composition comprises one selected from the group consisting of a Cas endonuclease, a pair of transcription activator-like effector nucleases (TALEN), and a pair of zinc-finger nucleases (ZFN). In some embodiments, the composition disclosed herein further comprises one or more viral delivery vectors containing the globin gene or the gene editing reagents. In some embodiments, the viral delivery vector comprises a lentivirus or an adeno-associated virus (AAV). In some embodiments, the viral delivery vector further comprises at least one surface modification that targets the vector to the HSCs. In some embodiments, the surface modification is an antibody linked to an external surface of the viral delivery vector, wherein the antibody targets hematopoietic stem cells, or precursors thereof.

In some embodiments, the composition disclosed herein further comprises at least one particle containing the globin gene and the gene editing reagents. In some embodiments, the at least particle comprises a plurality of lipid nanoparticles having the globin gene and the gene editing reagents embedded therein. In some embodiments, the plurality of lipid nanoparticles comprises at least: a first solid lipid nanoparticle comprising a segment of DNA that includes the globin gene; a second solid lipid nanoparticle that includes at least one Cas endonuclease complexed with a guide RNA (g RNA) that targets the Cas endonuclease to a locus within an alpha-globin gene cluster in chromosome 16. In some embodiments, the particle comprises one or a plurality of liposomes enveloping one or more of the globin gene and the gene editing reagents. In some embodiments, the globin gene is a zeta-globin gene or a zeta-globin coding region associated with at least one beta-globin regulatory region, at least one beta-globin intron, or both. In some embodiments, the zeta-globin gene includes a mutation in a 3′ sequence of a transcribed region. In some embodiments, when the composition is introduced into the fetus or patient after birth, or into the HSCs, the zeta-globin gene is transcribed into zeta-globin transcripts that are more stable than transcripts from a wild-type zeta-globin gene that is the same as the zeta-globin gene but that does not have the mutation in the 3′ sequence of the transcribed region. In some embodiments, after the composition is injected into the fetus, the fetus expresses the globin gene for at least a trimester.

A related embodiment of the composition according to the disclosure provides a composition for treatment of alpha thalassemia in a fetus, a patient after birth, or a cell thereof, the composition comprising: (a) a globin gene, wherein the globin gene is an alpha-globin gene, a zeta-globin gene, a zeta-globin gene associated with at least one beta-globin regulatory region, a zeta-globin gene associated with at least one beta-globin intron, or a zeta-globin gene associated with at least on beta-globin regulatory region and at least one beta-globin intron; and (b) gene editing reagents that, when the composition is introduced directly into the fetus or patient, or into HSCs derived from the fetus or patient, insert the globin gene into genomic material. In some embodiments, the patient is no older than one year of age. In some embodiments, the globin gene is an alpha-globin gene and the gene editing reagents comprise a first Cas9 ribonucleoprotein (RNP) that includes a first guide RNA (gRNA) and a second Cas9 RNP, wherein the first Cas9 RNP and the second Cas9 RNP bind to a locus within an alpha-globin gene cluster in chromosome 16 of the genomic material, and introduce the alpha-globin gene into the locus within the alpha-globin gene cluster.

In some embodiments, the gene editing reagents are targeted to a predetermined locus in the genomic material, wherein the locus is selected from: an alpha-globin gene cluster in chromosome 16; an intronic region of the beta-globin gene in chromosome 11, and an AAVS1, CCR5, CLYBL or hROSA26 genomic safe harbor. In some embodiments, the gene editing reagents comprise an mRNA that is translated into a gene editing nuclease selected from the group consisting of a Cas endonuclease, a pair of transcription activator-like effector nucleases (TALEN), and a pair of zinc-finger nucleases (ZFN). In some embodiments, the gene editing reagents comprise at least one guide RNA that targets the globin gene and a Cas endonuclease or nucleic acid encoding a Cas endonuclease, wherein the gene editing reagents, when introduced into a fetus, a patient after birth such as a patient no older than one year of age, or into cells obtained therefrom, introduce a change into a sequence within a globin gene within genomic material of the fetus, patient, cells, or progeny thereof, wherein the change activates or derepresses the globin gene. In some embodiments, the patient is no older than one year of age, such as a patient no older than three months of age. In some embodiments, the gene editing reagents introduce a mutation into a ZBTB7A binding site, a RREB1 binding site, or a NF-kB binding site in a repressor region in a 3′ end of the zeta-globin gene. In some embodiments, the gene editing reagents include CRISPR, TALENS, or ZFNs, and are included in the composition in a form that includes DNA, mRNA, or protein.

The disclosure also provides embodiments in which the composition is provided as a kit. In some embodiments, the kit includes additional reagents that promote integration of the globin gene into the genomic material, wherein the additional reagents include one or more of a polymerase, a ligase, dNTPs, a co-factor, and a topoisomerase. In some embodiments, the kit includes one or more surgical tools for delivering the globin gene and the gene editing reagents into the fetus, fetal circulation, or an organ of the fetus. In some embodiments, the kit further includes blood comprising alpha globin for transfusion into the fetus.

Another aspect of the disclosure is drawn to a method of treating alpha thalassemia, the method comprising: obtaining a sample comprising HSCs from a fetus or patient after birth, such as a patient no older than one year of age; using a composition disclosed herein to introduce the globin gene into the HSCs or progeny of the HSCs; and validating that the HSCs or the progeny express the introduced globin gene. In some embodiments, the method further comprises surgically accessing the fetus in the pregnant woman and introducing the HSC into fetal circulation by injection. Embodiments are contemplated that provide a method of treating alpha thalassemia, the method comprising: obtaining a sample comprising HSCs from a fetus or patient after birth by administering a composition comprising: (a) a globin gene, wherein the globin gene is an alpha-globin gene, a zeta-globin gene, a zeta- or alpha-globin gene associated with at least one beta-globin regulatory region, a zeta- or alpha-globin gene associated with at least one beta-globin intron, or a zeta- or alpha-globin gene associated with at least on beta-globin regulatory region and at least one beta-globin intron; and (b) gene editing reagents that, when the composition is introduced directly into the fetus or patient, or into HSCs derived from the fetus or patient, insert the globin gene into genomic material to introduce the globin gene into the HSCs or progeny of the HSCs. In some embodiments, the patient is no older than one year of age, including wherein the patient is no older than three months of age. In some embodiments, the method further comprises surgically accessing the fetus in the pregnant woman and introducing the HSC into fetal circulation by injection.

Embodiments include methods wherein the gene editing reagents, when introduced into a fetus or patient after birth such as a patient no older than one year of age, or into cells obtained therefrom, introduce a change into a sequence within a globin gene within genomic material of the fetus, patient, cells, or progeny thereof, wherein the change activates or derepresses the globin gene to modify the expression of the zeta-globin gene within the cells. In some embodiments, the gene editing reagents are introduced into fetal circulation, further wherein the modified globin gene is a modified zeta-globin gene that is resistant to gene silencing and is expressed persistently into at least a second trimester. Also contemplated are methods wherein the globin gene is inserted into the genomic material and expressed in the fetus or patient after birth, such as a patient no older than one year of age. In some embodiments, the methods further comprise introducing the cells or progeny thereof into fetal circulation by injection into an umbilical cord, placenta, liver, or heart of the fetus. In some embodiments, the gene editing reagents comprise at least one guide RNA and at least one Cas endonuclease or a nucleic acid encoding the Cas endonuclease. Embodiments are provided wherein the globin gene is an alpha-globin gene and the at least one guide RNA targets delivery of the alpha-globin gene to a predetermined locus in the genomic material, wherein the locus is selected from: an alpha-globin gene cluster in chromosome 16; an intronic region of beta-globin in chromosome 11, and a genomic safe harbor. In some embodiments, the globin gene is included as DNA and the gene editing reagents include at least one mRNA that, when introduced into the cells or fetus, is translated into a gene editing nuclease. In some embodiments, the gene editing reagents comprise at least a first Cas9 ribonucleoprotein (RNP) that includes a first guide RNA (gRNA) that binds the RNP to a locus within a globin gene cluster in the genomic material; and introduces the globin gene into the locus within the globin gene cluster.

Another aspect of the disclosure is drawn to a method for treating alpha thalassemia, the method comprising: introducing into a fetal cell, wherein the fetal cell comprises an HSC, RBC, or precursor thereof, or into circulation of a fetus, gene editing reagents that (i) increase production of alpha, zeta, or theta globin, (ii) decrease production of gamma globin, or (iii) decrease production of gamma globin and increase production of zeta globin. In some embodiments, the decreased production of gamma globin is due to a knockout mutation of gamma globin. In some embodiments, the zeta-globin gene is introduced into the fetal cell by insertion into the gamma globin gene, thereby decreasing production of gamma globin and increasing production of zeta globin. In some embodiments, the gene editing reagents include at least one composition selected from the group consisting of: a Cas endonuclease and a guide RNA; a nucleic acid encoding the Cas endonuclease and a nucleic acid encoding a guide RNA; a transcription activator-like effector nuclease (TALEN); a nucleic acid encoding the TALEN; a zinc-finger nuclease (ZFN); and a nucleic acid encoding the ZFN.

Some embodiments of this aspect of the disclosure provide a method wherein the gene editing reagents: introduce a mutation into a repressor region in a zeta-globin gene; and introduce a mutation into a 3′ sequence of a translated region of a zeta-globin gene. Some embodiments of the method further comprise administering gene editing reagents to the fetus to inhibit gene silencing of a zeta-globin gene and to increase persistence of zeta globin into at least a second trimester in the fetus, wherein the gene editing reagents include a Cas endonuclease gene and a DNA-sense guide RNA for introducing a mutation into a repressor region in the zeta-globin gene or for introducing a mutation into a 3′ sequence of a transcribed region of the zeta-globin gene. In some embodiments, the fetal cell is a hematopoietic stem cell (HSC), wherein the gene editing reagents introduce an activating mutation in the zeta-globin gene of the HSC ex vivo, and wherein the mutated HSC or progeny thereof is delivered to the fetal circulation by injection into the fetus, umbilical cord, or placenta.

Yet another aspect of the disclosure is a composition for treatment of alpha thalassemia in a fetus, or patient after birth such as a patient no older than one year of age, the composition comprising a globin gene, wherein the globin gene is an alpha-globin gene, a zeta-globin gene, a zeta- or alpha-globin gene associated with at least one beta-globin regulatory region, a zeta- or alpha-globin gene associated with at least one beta-globin intron, or a zeta- or alpha-globin gene associated with at least on beta-globin regulatory region and at least one beta-globin intron; and gene editing reagents that when introduced into a fetus or patient after birth, such as a patient no older than one year of age, or into cells obtained therefrom, introduce a change into a sequence within a globin gene within genomic material of the fetus, patient, cells, or progeny thereof, wherein the change activates or derepresses the globin gene. A related aspect is drawn to a composition for treatment of alpha thalassemia in a fetus, or a patient after birth, such as a patient no older than one year of age, or a cell thereof, the composition comprising: (a) a globin gene, wherein the globin gene is an alpha-globin gene, a zeta-globin gene, a zeta- or alpha-globin gene associated with at least one beta-globin regulatory region, a zeta- or alpha-globin gene associated with at least one beta-globin intron, or a zeta- or alpha-globin gene associated with at least on beta-globin regulatory region and at least one beta-globin intron; and (b) gene editing reagents that, when the composition is introduced directly into the fetus or patient, or into HSCs derived from the fetus or patient, insert the globin gene into genomic material. In some embodiments of either of these related aspects, the gene editing reagents are provided in a viral vector or non-viral particle, wherein the viral vector or the non-viral particle optionally includes one or more targeting molecules that target the viral vector or the non-viral particle to fetal cells or the HSCs. In some embodiments, the gene editing reagents include a Cas endonuclease, or nucleic acid encoding the Cas endonuclease, and one or more guide RNAs that target the globin gene. In some embodiments, the globin gene is a zeta- or alpha-globin gene and the gene editing reagents introduce a mutation into the zeta- or alpha-globin gene. In some embodiments, the gene editing reagents introduce a mutation into at least one repressor region in a zeta- or alpha-globin gene or wherein the gene editing reagents activate a zeta- or alpha-globin gene. In some embodiments, the repressor region is a ZBTB7A binding site, a RREB1 binding site, or includes a NF-kB binding region in a 3′ end of the zeta-globin gene.

In some embodiments, after the change is introduced within the sequence of the globin gene, the zeta-globin gene is not silenced at the end of a first trimester, and the zeta globin is expressed beyond the first trimester and optionally after birth. In some embodiments, the gene editing reagents introduce a mutation into a 3′ sequence of a transcribed region of a zeta-globin gene. In some embodiments, after the change is introduced within the sequence of the globin gene, the mutated zeta-globin gene is transcribed into modified transcripts, wherein the modified transcripts persist in fetal cells for longer than similar but unmodified transcripts. In some embodiments, the gene editing reagents introduce a mutation into at least one repressor region in a zeta-globin gene and introduce a mutation into a 3′ sequence of a transcribed region of a zeta-globin gene. In some embodiments, the composition further includes, or is provided in a kit that also includes, blood comprising alpha globin for transfusion to the fetus. In some embodiments, the gene editing reagents include CRISPR, TALENS, or ZFNs, and are included in the composition in a form that includes DNA, mRNA, or protein. In some embodiments, the gene editing reagents further introduce a promoter or a transcription factor binding site to increase transcription of the zeta-globin gene. In some embodiments, the composition is provided in a kit that also includes surgical tools for injection of the composition into the fetus.

Still another aspect of the disclosure is a method of treating alpha thalassemia, the method comprising: obtaining a sample comprising cells from the fetus or patient after birth, wherein the cells comprise HSCs or precursors thereof; using a composition of claim 29 to modify a zeta-globin gene within the cells; and validating that the cells or progeny thereof express the modified zeta-globin gene. Some embodiments of the method further comprise surgically accessing the fetus in the pregnant woman and introducing the cells or progeny thereof into fetal circulation, whereby the modified zeta-globin gene in the fetus is resistant to gene silencing and is expressed persistently into at least a second trimester.

Another aspect of the disclosure is directed to a method of treating alpha thalassemia, the method comprising: introducing, into a fetus or postnatal patient, or into cells obtained therefrom wherein the cells include HSCs or precursors thereof, a globin gene and gene editing reagents, whereby the gene editing agents cause insertion of the globin gene into genomic material and cause the globin gene to be expressed. Some embodiments of the method further comprise introducing the cells or progeny thereof into fetal circulation by injection into an umbilical cord, placenta, liver, or heart of the fetus. In some embodiments, the gene editing reagents include at least one Cas endonuclease or a nucleic acid encoding the Cas endonuclease and a guide RNA. Some embodiments of the method further comprise surgically accessing the fetus in vivo in a pregnant subject and injecting the reagents into the fetal circulation, fetal liver, or placenta in vivo.

In some embodiments, the globin gene is an alpha-globin gene and the gene editing reagents include one or more guide RNAs that target delivery of the alpha-globin gene to a predetermined locus in the genomic material, wherein the locus is selected from: an alpha-globin gene cluster in chromosome 16; an intronic region of beta-globin in chromosome 11, and a genomic safe harbor. In some embodiments, the globin gene is contained within a lentiviral or adeno-associated virus vector. In some embodiments, the globin gene is an alpha-globin gene or a zeta-globin gene. Some embodiments of the method further comprise performing the introducing step while delivering a therapeutic blood transfusion with blood that includes alpha globin. In some embodiments, the nucleic acid encoding the Cas endonuclease and the globin gene are packaged using one or more lentiviral or adeno-associated virus (AAV) vector. In some embodiments, the globin gene is included as a segment of DNA that also includes one or more of a promoter, a fluorescent protein, an SV40 sequence, and a poly(A) sequence. In some embodiments, the globin gene is included as DNA and the gene editing reagents include at least one mRNA that, when is introduced into the cells or fetus, is translated into a gene editing nuclease by a ribosome. In some embodiments, the gene editing reagents comprise a first Cas9 ribonucleoprotein (RNP) that includes a first guide RNA (gRNA) and a second Cas9 RNP, wherein the first Cas9 RNP and the second Cas9 RNP bind to a locus within a globin gene cluster in the genomic material, such as binding by binding to a locus within a zeta- or alpha-globin gene; and introduces the globin gene, e.g., the zeta- or alpha-globin gene, into the locus within the globin gene cluster.

Another aspect of the disclosure provides a method for treating alpha thalassemia, the method comprising: introducing into a fetal cell, wherein the fetal cell comprises an HSC, RBC, or precursor thereof, or into circulation of a fetus, gene editing reagents that (i) increase production of alpha, zeta, or theta globin, (ii) decrease production of gamma globin, or (iii) decrease production of gamma globin and increase production of zeta globin. In some embodiments, the decreased production of gamma globin is due to a knockout mutation of gamma globin. In some embodiments, zeta globin production is increased by activating an endogenous zeta-globin gene or by introducing a zeta-globin gene into the fetal cell. In some embodiments, the zeta-globin gene is introduced into the fetal cell by insertion into the gamma globin gene, thereby decreasing production of gamma globin. In some embodiments, the gene editing reagents include at least one composition selected from the group consisting of: a Cas endonuclease and a guide RNA; a nucleic acid encoding the Cas endonuclease and a nucleic acid encoding a guide RNA; a transcription activator-like effector nuclease (TALEN); a nucleic acid encoding the TALEN; a zinc-finger nuclease (ZFN); and a nucleic acid encoding the ZFN. In some embodiments, the gene editing reagents introduce a change into a sequence within a zeta-globin gene. In some embodiments, the gene editing reagents introduce a mutation into a repressor region in a zeta-globin gene. In some embodiments, the repressor region includes a NF-kB binding region in a 3′ end of the zeta-globin gene. In some embodiments, the gene editing reagents introduce a mutation into a 3′ sequence of a translated region of the zeta-globin gene. In some embodiments, after the introducing step, the mutated zeta-globin gene is transcribed into modified transcripts, wherein the modified transcripts persist for longer than similar but unmodified transcripts. In some embodiments, the gene editing reagents: introduce a mutation into a repressor region in a zeta-globin gene; and introduce a mutation into a 3′ sequence of a translated region of a zeta-globin gene. some embodiments of the method further comprise performing the introducing step while delivering to the fetus a therapeutic blood transfusion with blood that includes alpha globin. In some embodiments, the gene editing reagents are provided at least in part in a viral vector or non-viral particle for delivery. In some embodiments, the gene editing reagents further introduce a promoter or a transcription factor binding site to increase transcription of the zeta-globin gene. Some embodiments of the method further comprise inhibiting gene silencing of a zeta-globin gene and increasing persistence of zeta globin into at least a second trimester in the fetus by injecting into the fetus via a needle the gene editing reagents, wherein the gene editing reagents include a Cas endonuclease gene and a DNA-sense guide RNA packaged in a viral vector, wherein, when the gene editing reagents are expressed in the fetus, the gene editing reagents introduce a mutation into a repressor region in the zeta-globin gene or introduce a mutation into a 3′ sequence of a transcribed region of the zeta-globin gene. In some embodiments, the method includes: obtaining the HSC from the fetus, cord blood, or from the patient after birth; performing the introducing step ex vivo to introduce the change into the zeta-globin gene in the HSC, whereby the production of zeta globin in the HSC is increased; and delivering the modified HSC, or progeny thereof, into fetus circulation by injection into the fetus, umbilical cord, or placenta.

Other features and advantages of the disclosed subject matter will be apparent from the following detailed description and figures, and from the claims.

### BRIEF DESCRIPTION OF THE DRAWING

FIG. 1 diagrams a method for treating ATM by stem cell (HSC) transplant.

FIG. 2 shows steps of the method according to certain embodiments.

FIG. 3 shows a target for introducing a globin gene.

FIG. 4 shows introducing gene editing reagents into HSCs.

FIG. 5 diagrams an in vivo method for treating ATM with gene editing reagents.

FIG. 6 shows delivering gene editing reagents to a fetus in utero.

FIG. 7 shows a Cas endonuclease complexed with a guide RNA.

FIG. 8 shows a plasmid for treating alpha thalassemia by gene editing.

FIG. 9 shows an mRNA for a gene editing protein.

FIG. 10 shows a composition for treating ATM by gene replacement.

FIG. 11 shows a composition useful for editing a globin gene.

FIG. 12 shows a viral vector with gene editing reagents that target a globin gene.

FIG. 13 shows a liposome for delivery of a composition of the disclosure.

FIG. 14 is a cartoon of a lipid nanoparticle (LNP).

FIG. 15 shows a kit according to embodiments of the disclosure.

FIG. 16 shows globin chain contribution to embryonic, fetal, and adult hemoglobin.

FIG. 17 diagrams a method for treating ATM by decreasing gamma globin.

FIG. 18 reveals that engineered erythroid cells model alpha-thalassemia major in vitro. (A) Schematic depicting the CRISPR-Cas9 gene editing strategy employed to generate HUDEP2 cells carrying the most common ATM deletion (ATM^{ΔSEA}). (B) PCR amplification of genomic DNA from edited cell clones defined two clones as homozygous and three clones as heterozygous for the d ATM^{ΔSEA} deletion. (C) Western Blot probing for the presence of alpha- and zeta-globin chains in wild-type (WT) and ATM^{ΔSEA} clones. GAPDH protein levels were determined as a loading control. (D) Western Blot probing for beta-globin chains. Samples were denatured without reducing agent before electrophoresis, allowing disulfide bonds to persist. Beta-globin disulfide dimers were present in homozygous ATM^{ΔSEA} cells. GAPDH protein levels were determined as a loading control. (E-F) ATM^{ΔSEA} cells show reduced alpha-globin (HBA) and elevated zeta-globin (HBZ) mRNA levels. Expression levels were determined by qPCR and normalized to expression levels of the RPL13A gene.

FIG. 19 reveals that alpha and zeta globin can be expressed at high levels from a lentiviral construct. (A) Schematic of the workflow. Lentiviral plasmids were cloned to contain the coding sequences of zeta- or alpha-globin driven by a truncated beta-globin enhancer and the beta-globin promoter. Alpha- and zeta-globin introns and 3′UTRs were replaced with beta-globin introns (second intron truncated) and beta-globin polyA tail (3′UTR). Lentivirus was produced in HEK293T cells and HUDEP2 cells were transduced with varying amounts of lentivirus (lentiviral supernatant); (B) Transduced HUDEP2 cells express alpha- or zeta-globin at high levels. Western Blot showing expression levels of zeta globin and alpha globin in transduced cells. Expression levels correlate with viral titer used for transduction. Expression of GAPDH was used as a loading control.

FIG. 20 discloses strategies to replace the alpha-globin gene in cells with alpha-thalassemia major. (A) Schematic showing the outline of the knockin strategy. Top: schematic of the alpha (chromosome 16) and beta-globin (chromosome 11) loci. Enlarged: Schematic of the beta-globin gene. Using a nuclease, a double-stranded break was made in either intron 1 or intron 2 of the HBB gene. Donor DNA deliverable by AAV6 contains homology arms to the 5′UTR of beta-globin and 400 bp downstream of the cut site. The DNA donor contains alpha-globin, including its introns and 3′UTR. When alpha-globin is successfully knocked into the HBB locus, the HBB gene is dysfunctional and alpha-globin protein is made instead. In the case of no knockin, indels will form in the introns of HBB, which will not affect expression of the gene that can thus still produce functional beta-globin. Together, the gene products form functional adult hemoglobin (HbA). (B) Two potential gRNA, one in intron 1 and one in intron 2, were identified as efficient cutters (guide RNAs 7 and 13). (C) Editing with these gRNAs did not affect beta-globin protein levels, as determined by Western Blot. (D) Expression levels from (C) were quantified and normalized against a GAPDH loading control.

FIG. 21 shows that disrupting HBZ repressor elements results in elevated HBZ expression levels. (A) Schematic of an example of two HBZ repressors binding to the zeta-globin promoter. Editing the repressor binding sites with a nuclease resulted in loss of the binding motif and derepression of zeta-globin. (B) mRNA expression levels of HUDEP2 cells that were edited with gRNAs targeting either the RREB1 binding site or the ZBTB7A binding site in the HBZ promoter. RNA levels were normalized to a reference gene (RLP13A). (C) Schematic of the workflow to discover new HBZ repressor elements in an unbiased manner. A lentiviral gRNA library tiling the entire HBZ and HBA globin locus is produced in HEK293T cells. HUDEP2 cells with stable Cas9 expression were transduced with the library, and differentiated cells were sorted into HBZ high- and low-expression bins. Relative abundance of gRNAs in each bin identified genetic elements affecting zeta-globin repression.

### DETAILED DESCRIPTION

The disclosure provides methods, kits, and compositions that employ gene therapy or gene editing for the treatment of ATM. Compositions and methods of the disclosure are useful to correct for an ATM associated genotype by introducing or increasing expression a globin gene or, in some cases, downregulating an over-produced globin gene.

One set of embodiments of the disclosure involves ex vivo hematopoietic stem cell (HSC) treatment and HSC transplant. In ex vivo embodiments, cells are obtained from the fetus. Cells can be removed from the fetus such as fetal HSCs or cells that can be differentiated into HSCs such as amniotic fluid cells. The HSCs may be expanded and/or treated ex vivo with gene editing reagents to introduce, modify, or regulate expression of a globin gene. For example, an alpha-globin gene may be inserted into the genome or stably expressed via in the HSCs using an expression vector. The HSCs are then delivered to the fetus (e.g., via surgical access and injection into fetal circulation) or to the subject in a postnatal period. The modified HSCs then naturally circulate and express the globin gene that was introduced, modified, or regulated. Where, for example, the globin gene is an exogenous alpha-globin gene that was inserted into a genome in the HSCs, the HSC then express alpha globin, whereby the ATM is treated.

In other embodiments, the disclosure involves in vivo delivery, to a subject, gene editing reagents that will introduce, modify, or regulate expression of a globin gene in the subject. The subject is preferably a fetus in utero although the subject may be treated after birth. For example, optionally using a viral vector or a non-viral particle, a genome editing tool such as a CRISPR system can be delivered, along with a copy of a globin gene, into circulation in the fetus. Gene editing systems by their nature have human-designed and human-made sequences that make then unique and imbue the gene editing reagents with a specific associated function. In CRISPR systems, the guide RNAs have unique sequences, whereas in ZFNs and TALENs the protein sequences are unique and application-specific. Those sequences in the gene editing reagents are cognate to predetermined targets within nucleic acid. For fetal in vivo treatment of ATM, the gene editing reagents target a predetermined target within an alpha-globin gene cluster in chromosome 16 within fetal HSCs, red blood cells (RBCs), or their precursors or progeny, or insert a globin gene into a locus where it will be expressed such as the alpha-globin gene cluster, a beta-globin gene cluster on chromosome 11, or a genomic safe harbor. The circulating fetal cells then express in the inserted globin gene, whereby the ATM is treated.

Both the ex vivo and in vivo approaches just summarized can be used to treat ATM by one or a combination of several strategies including increasing production of alpha globin, increasing production of zeta globin, decreasing production of gamma globin, and others, which are discussed herein below.

Specific applications include the use of viral vectors or other delivery tools to deliver gene editing reagents or to express the relevant genes using gene therapy. Relevant viral vectors include adeno-associated viral (AAV) vectors, which may bear one or any number of capsid proteins, such as aav6 or others that target HSCs or other relevant progeny populations (e.g., to edit RBCs), lentiviral vectors, or other retroviral vectors. Non-viral systems including but not limited to lipid nanoparticles or other nanoparticles can be used to deliver the genes or the gene editing reagents. Such vectors or particles may be targeted to HSCs or RBCs/RBC precursors using, e.g., antibodies.

While ATM is the condition with four mutated alpha-globin genes located in cis, other alpha thalassemia variants can also be treated with these strategies (including but not limited to Hb H disease or Hb Constant spring). For example, patients with anemia secondary to milder alpha thalassemia variants may be treated using compositions and methods of the disclosure. The disclosure provides several gene therapy or editing strategies and associated methods and compositions that can address the lack of the alpha gene and that can also be applied to increasing the production of any globin gene such as the zeta gene. Gene therapy can be used to insert a working alpha-globin or zeta-globin gene.

### I. Ex Vivo Methods Addressing HSC

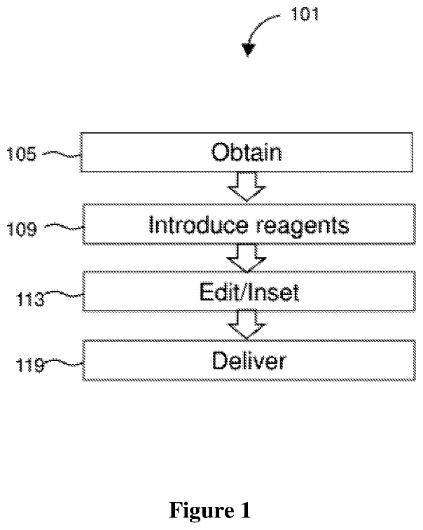

FIG. 1 diagrams a method 101 for treating alpha thalassemia by hematopoietic stem cell (HSC) transplant. The method 101 includes obtaining 105 cells that will be treated. Cells can be removed from the fetus such as fetal HSCs or cells that can be differentiated into HSCs such as amniotic fluid cells. HSCs can also be obtained from cord blood or from the patient at any time after birth. Gene editing reagents are introduced 109 into the cells. The cells may be expanded and are treated with the gene editing reagents to introduce, modify, or regulate expression of a globin gene. For example, a globin gene may be inserted into the genome or stably expressed in the cells using an expression vector. Alternatively, an endogenous globin gene may be edited to regulate its expression. These strategies are each discussed in greater detail herein below. The gene editing reagents either edit a globin gene or insert 113 a globin gene into the cells to promote expression of the gene in the cells. In some embodiments, the edited/modified cells are packaged and stored for later use. For example, where there is no pressing or instantaneous need to treat a living fetus or in other (e.g., research applications), the cells may be stored for later future use or research, e.g., collected in a blood collection tube such as the blood collection tube sold under the trademark VACUTAINER by BD (Franklin Lakes, N.J.), processed using a Ficoll gradient or purification of CD34+ HSCs, frozen in a medium containing DMSO, and stored in liquid nitrogen in cryovial tubes. In preferred embodiments, the cells are delivered 119 to fetus. For example, a surgical incision may be made in the mother's abdomen and a needle used to inject the cells into the fetus.

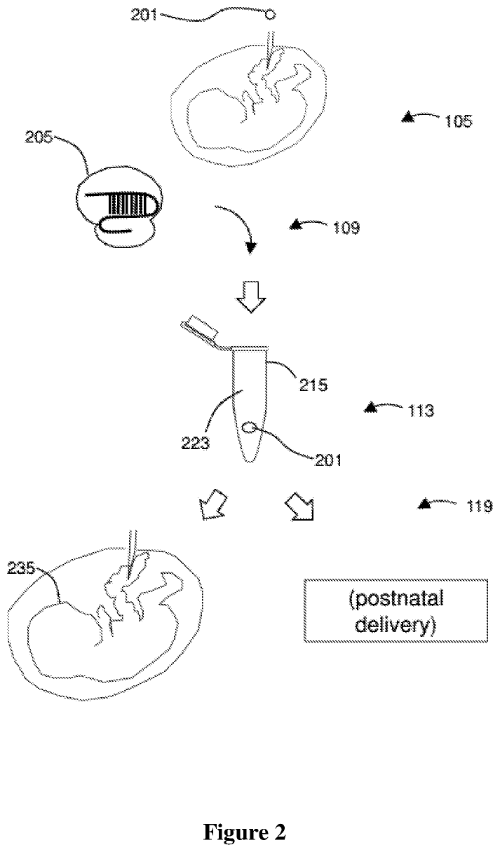

FIG. 2 shows steps of the method 101 according to certain embodiments. In this depicted embodiment, cells 201 (e.g., fetal or postnatal HSCs) are obtained 105. The gene editing reagents 205 include a CRISPR system. The gene editing reagents 205 are introduced 109 into the cells 201. The gene editing reagents 205 introduce 113 a globin gene or edit a globin gene within the cells 201. The gene editing steps may be performed in a suitable media 223 such as blood or a buffer or solution, all within a suitable container 215 such as a well of a multi-well plate, test tube, or micro-centrifuge tube such as the tube sold under the trademark EPPENDORF® by Fisher Scientific Co. L.L.C. (Pittsburgh, Pa.). Some embodiments include editing a globin gene with the cells 201. Certain embodiments include introducing 109 a globin gene with gene editing reagents 205 into an HSC, whereby the gene editing agents cause insertion of the globin gene into genomic material and cause a globin to be expressed. The modified cells 201 may be delivered 119 into a placenta or fetus 235. A laparotomy may be performed to access the uterus. Then, the method may include using ultrasound guidance to access the fetus. The cells are then delivered into fetal circulation by injection into the umbilical vein, placenta, fetal heart or liver, using a spinal needle. Other embodiments are within the scope of the disclosure. For example, as discussed below, in the fetus, in vivo therapy can be performed by injecting the gene therapy or gene editing reagents directly into the fetus, either into the bloodstream or into the fetal liver or heart or into the placenta to treat hematopoietic stem cells (HSCs)). Here, using the method 101, ex vivo therapy may be performed in the fetus by removing cells from the fetus (such as HSCs, or cells that can later be differentiated into HSCs such as amniotic fluid cells) or cord blood/mobilized peripheral blood/bone marrow HSCs obtained from the patient after birth. The relevant gene(s) can be expressed either in HSCs, or in cells that can ultimately become working red blood cells (RBCs) such as erythrocytes or erythrocyte precursors. In further related embodiments, after birth, in vivo gene therapy or editing can also be performed. For example, HSCs derived from cord blood, bone marrow, or mobilized peripheral blood can be edited ex vivo and transplanted into the patient.

These genes can be under the control of an alpha-globin locus control region (LCR) such as HS-40 or the beta-globin LCR, or another suitable promoter that will ensure correct level of expression of the alpha or zeta chain. See Chen, 1997, Analysis of enhancer fusion of the HS-40 core sequence of the human alpha-globin cluster, Nucleic Acids Res 25(14):2917-2922, incorporated by reference. Gene delivery may be performed by various means including but not limited to transfection of the cells using gene-containing plasmids, using nanoparticles to deliver the genes, or viral vectors such as lentiviral or AAV vectors. Using such methods, a globin gene may be inserted into a genome, delivered in an expression vector, or edited to affect expression.

In certain preferred embodiments, ATM is treated by inserting an alpha-globin gene into the genome.

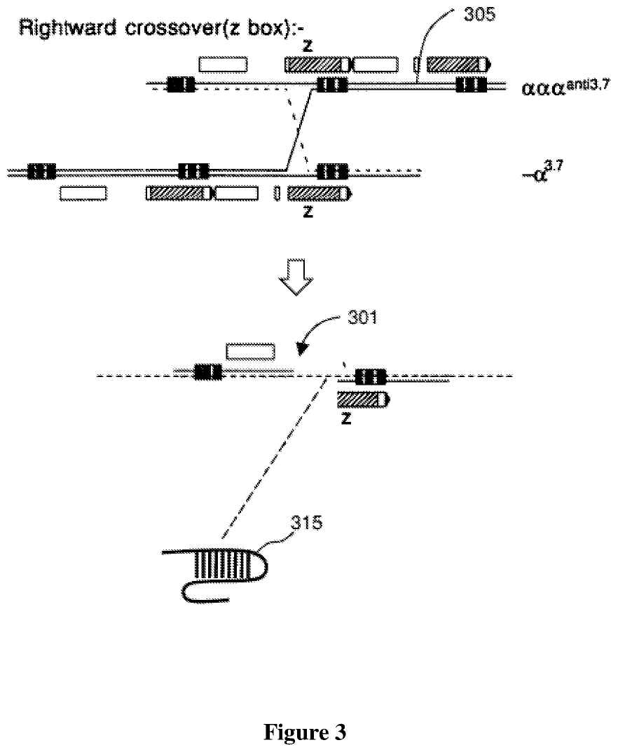

FIG. 3 shows a target 301 for introducing a globin gene. The top panel is a cartoon of two copies of an alpha-globin gene cluster 305, with lines showing a type of crossover event that can lead to loss of a copy of a globin gene. The middle panel illustrates a hypothetical segment of a chromosome with a deleted alpha-globin gene and shows a target 301 location for insertion of a replacement gene. The alpha-globin gene cluster spans from about base pair 140,000 to about 180,000 of chromosome 16. Using this information, one of skill in the art can prepare or order gene editing reagents useful to insert a copy of an alpha-globin gene at the target 301. For example, one may access the sequence of the alpha-globin gene cluster (base pairs 140,000 to 180,000 of chromosome 16) from the published human genome and scan that sequence (e.g., with a computer program) to search for and identify targets suitable for editing with a gene editing reagent 205.

For example, where the gene editing reagents include a CRISPR system that uses a Cas9 endonuclease from Streptococcus pyogenes (spCas9) complexed with a guide RNA 315 as a ribonucleoprotein RNP, one may design the guide RNA 315 to include a 20-base targeting sequence that is complementary to a suitable target in the gene cluster 305 (or within a few hundred or thousand bases of the gene cluster 305). For spCas9, the target is a sequence that matches 5′-20 bases-protospacer adjacent motif (PAM)-3′, where the PAM is NGG. To insert an exogenous gene, two spCas9 RNPs are used, with a pair of guide RNAs 315 that flank the target 301. The RNPs bind to their cognate targets in the cluster 305 and introduce double stranded breaks. The exogenous gene being inserted may be provided with ends that are homologous to sequences flanking the target 301 to invoke the cell's endogenous homology-directed repair response, which “repairs” the genome by inserting the exogenous DNA segment. See How, 2019, Inserting DNA with CRISPR, Science 365(6448):25 and Strecker, 2019, RNA-guided DNA insertion with CRISPR-associated transposases, Science 365(6448):48, both incorporated by reference. Thus, in the depicted embodiment, the alpha-globin gene is inserted into its usual, or natural, position (where it has been deleted in the patient with alpha thalassemia mutations) using gene editing reagents. However, the alpha-globin gene may be entered at some other locus.

The method 101 may be performed with any suitable gene editing reagents 205 including CRISPR systems, ZFNs, or TALENs. The composition of the gene editing reagents 205 is unique in that they are cognate to intended targets such as the alpha-globin gene cluster or its associated locus control region. Thus the gene editing reagents 205 can be designed and synthesized or ordered by making reference to the target gene cluster.

The gene cluster contains 1 embryonic zeta- and 2 alpha-globin genes arranged in the order of 5′-zeta2-alpha2-alpha1-3′ on each chromosome 16. There are 4 pseudogenes within the alpha-globin gene cluster. Since each individual has 2 chromosomes 16, there are usually a total of 4 functional alpha-globin genes. Overall, the combined production of alpha-globin chains from these 4 α-globin genes is approximately equivalent to that of the beta-globin chains derived from the 2 beta-globin genes on chromosome 11. The number of alpha-globin genes per chromosome 16 can range from 0 to 4, owing to unequal crossing over between misaligned alpha-globin gene clusters and other recombination events. Therefore the total number of alpha-globin genes an person may have can range from 0 to as many as 7 or 8. Whereas the alpha2- and alpha1-globin genes encode identical α-globin chains of 141 amino acid residues, the alpha2-globin gene accounts for twice the alpha-globin chains produced relative to the alpha1-globin gene, likely owing to the effect of different promoter sequences that are proximal to the coding sequences. See Waye, 2001, The alpha-globin gene cluster: genetics and disorders, Clin Invest Med 24(2):103-9, incorporated by reference. The alpha1-globin, aka HBA1, gene provides instructions for making (i.e., a sequence that is transcribed and then translated into) a protein called alpha-globin. This protein is also produced from a nearly identical gene called HBA2. The HBA1 gene is located at base pairs 176,680 to 177,522 on chromosome 16.

The alpha-globin locus control region (at 87808 . . . 152854 on chromosome 16) regulates developmental stage- and erythroid lineage-specific expression of the HBZ (hemoglobin, zeta), HBA2 (hemoglobin, alpha 2), HBA1 (hemoglobin, alpha 1) and HBQ1 (hemoglobin, theta 1) genes within the alpha-globin gene cluster. This region has properties of a locus control region (LCR) in that it can confer high-level and chromosomal position-independent expression on members of the alpha-globin gene cluster in a transgene assay, but unlike other LCRs, such as that regulating the beta-globin gene cluster, it lacks the ability to confer copy number-dependent expression on the linked genes. This region overlaps the NPRL3 (NPR3-like, GATOR1 complex subunit) gene, which is transcribed in the opposite orientation compared to the downstream alpha-globin genes. This regulatory region is characterized by multiple erythroid-specific DNase I hypersensitive sites, including HS-48, HS-40, HS-33, HS-10 and HS-8, where the HS-40 site represents an enhancer and is the major cis-acting regulatory element. HS-40 binds transcription factors and mediates looping with the promoters of the alpha-globin genes during erythroid development. The HS-40 element has also been used to enhance erythroid expression of beta-globin family members in gene therapy vectors for beta-chain hemoglobinopathies. Mutations in this regulatory region result in alpha thalassemias and alpha hemoglobinopathies.

In another strategy, a globin gene (e.g., the alpha-globin gene) and a suitable promoter (such as the alpha or beta LCR as described above) can be expressed in another locus, such as a safe harbor, using gene editing reagents. In this depicted embodiment. the globin gene is an alpha-globin gene and the gene editing reagents include one or more guide RNAs 315 that target delivery of the alpha-globin gene to a predetermined locus, or target 301, in the genomic material. The target 301 is in the alpha-globin gene cluster in chromosome 16. Alternatively, the target could be in a beta-globin gene cluster in chromosome 11 or a genomic safe harbor.

A globin gene can be inserted into its target 301 within cells, e.g., in the ex vivo method 101.

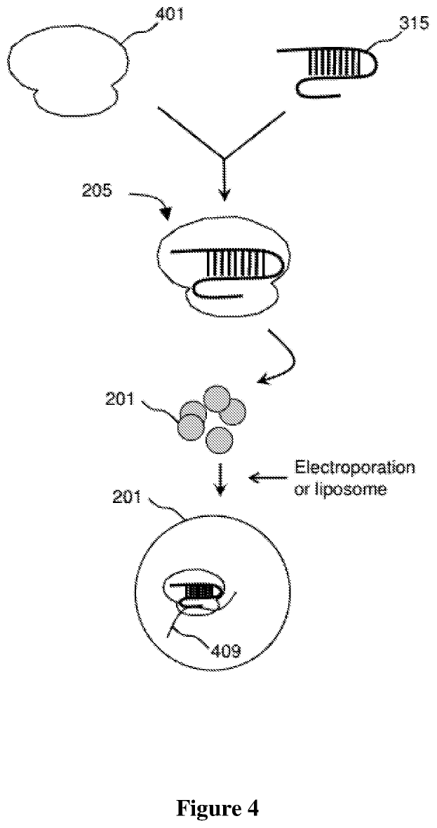

FIG. 4 shows introducing gene editing reagents into HSCs 201. The guide RNAs 315 are designed with reference to a human genome sequence and synthesized or obtained, e.g., ordered from a custom oligonucleotide synthesis company such as Integrated DNA Technologies (Coralville, Iowa). A Cas endonuclease 401 is obtained or synthesized and the gRNA 315 is complexed with the Cas endonuclease 401 to form an RNP for use as the gene editing reagents 205. The gene editing reagents 205 are introduced into the cells 201 where they edit genomic material 409 to insert a globin gene (e.g., alpha-globin gene) into the target 301 in the alpha-globin cluster on chromosome 16 or other suitable site. This depicted embodiment includes introducing RNP into target cells in an ex vivo procedure, which may be promoted via electroporation or suitable packaging such as a liposome or other particle. Other embodiments are within the scope of the disclosure.

### II. In Vivo Methods Addressing a Fetus

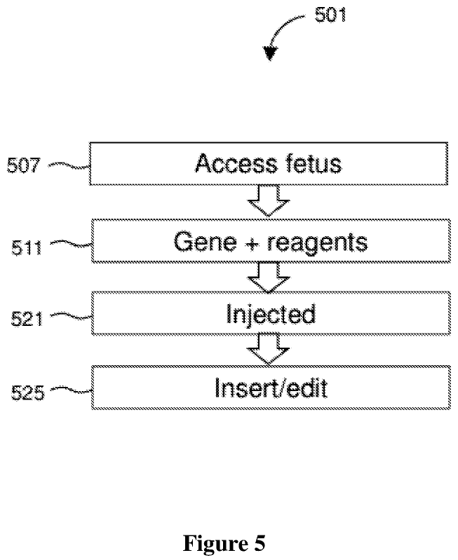

FIG. 5 diagrams an in vivo method 501 of directly treating a fetus with gene editing reagents to treat ATM. In the method 501, the fetus 507 is accessed using ultrasound guidance in the umbilical vein. A small blood sample is obtained to measure Hb levels. A small (1-2 cc) blood transfusion is then infused to ensure that the needle position is intact. Gene editing reagents are obtained 511 along with, optionally, a copy of a globin gene to be inserted. The gene editing reagents are then injected 521, which may be followed by the remainder of the RBC transfusion (such that the total volume is blood corrects the fetal anemia). Optionally, the gene therapy/editing reagents could be mixed with markers that are visible on ultrasound to ensure that the full volume enters the fetal circulation. The gene editing reagents then insert or edit 525 a globin gene in circulating cells in the fetus.

For these patients, additional modifications of the gene delivery reagents to specifically target fetal HSCs may be included. For example, the gene editing reagents (e.g., through their viral vector coats or carrier particle surfaces) may have antibodies or other binding proteins that target specific surface markers on fetal HSCs. Viral capsid proteins may be modified to improve transduction of fetal HSCs. For ex vivo applications, selection of fetal HSCs (from a fetal blood sample or placental biopsy) and expansion of those cells (using reagents that are developed to amplify fetal HSC expansion) may be developed.

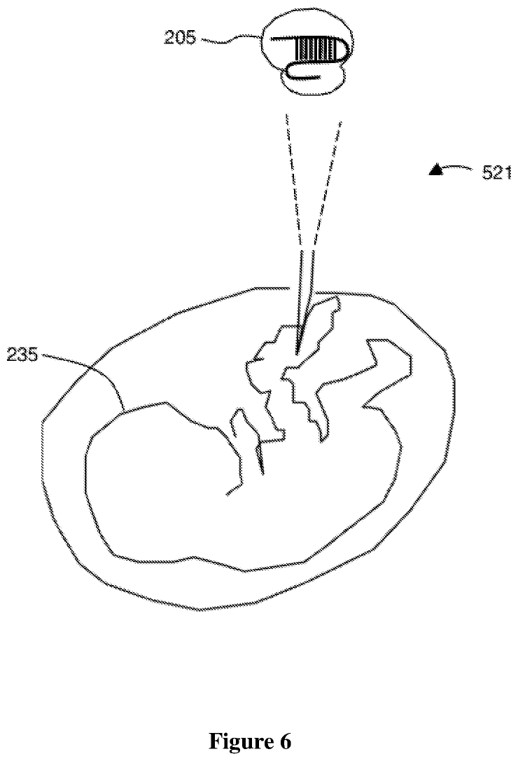

FIG. 6 shows delivering 521 gene editing reagents 205 to a fetus 235 in utero, in vivo. The delivery 521 may be done by surgically accessing the fetus in vivo in a pregnant subject and injecting the reagents into fetal circulation (e.g., via injection into the umbilical cord), the fetal liver or heart, or the placenta in vivo. The gene editing reagents 205 may be used to insert a globin gene into a genome of a cell in the fetus (e.g., within fetal HSCs, RBCs, precursors, or progeny), or to edit such a gene, or deliver an expression vector to the cell to express a globin gene.

For example, the fetus or patient after birth can be treated in vivo with a lentiviral vector or AAV vector expressing the alpha- or zeta-globin gene with a suitable promoter. Thus embodiments, of the method 501 include in utero gene therapy using, e.g., a lentiviral vector or an adeno-associated virus (AAV) vector. For background, see Han, 2007, Fetal gene therapy of alpha-thalassemia in a mouse model. PNAS 104:9007-11, incorporated by reference.

### III. Gene Editing Reagents

Compositions and methods of the disclosure include gene editing reagents 205 for introducing or editing a globin gene. Gene editing reagents generally include a kind of programmable nuclease, which generally refers to an enzyme that cleaves nucleic acid that can be or has been designed or engineered by human contribution so that the enzyme targets or cleaves the nucleic acid in a sequence-specific manner. Programmable nucleases include zinc-finger nucleases (ZFNs), transcription activator-like effector nucleases (TALENs) and RNA-guided nucleases such as the bacterial clustered regularly interspaced short palindromic repeat (CRISPR)—Cas (CRISPR-associated) nucleases or Cpf1. Programmable nucleases also include PfAgo and NgAgo.

ZFNs cut genetic material in a sequence-specific matter and can be designed, or programmed, to target specific viral targets. A ZFN is composed of two domains: a DNA-binding zinc-finger protein linked to the FokI nuclease domain. The DNA-binding zinc-finger protein is fused with the non-specific FokI cleave domain to create ZFNs. The protein will typically dimerize for activity. Two ZFN monomers form an active nuclease; each monomer binds to adjacent half-sites on the target. The sequence specificity of ZFNs is determined by ZFPs. Each zinc-finger recognizes a 3-bp DNA sequence, and 3-6 zinc-fingers are used to generate a single ZFN subunit that binds to DNA sequences of 9-18 bp. The DNA-binding specificities of zinc-fingers is altered by mutagenesis. New ZFPs are programmed by modular assembly of pre-characterized zinc fingers. ZFN may be used to cut viral nucleic acid. Briefly, the ZFN method includes introducing into the target cell a ZFN or a vector (e.g., plasmid) encoding a targeted ZFN and, optionally, at least one accessory polynucleotide. See, e.g., U.S. Pub. 2011/0023144 to Weinstein, incorporated by reference. The cell includes target sequence. The cell is incubated to allow expression of the ZFN, wherein a double-stranded break is introduced into the targeted sequence by the ZFN. In some embodiments, a donor polynucleotide or exchange polynucleotide is introduced. Target DNA along with exchange polynucleotide may be repaired by an error-prone non-homologous end-joining DNA repair process or a homology-directed DNA repair process. The latter may be promoted by supplying a globin gene in a DNA fragment with ample (e.g., a few hundred bases) overlap to the target 301 at the ends.

Typically, a ZFN comprises a DNA binding domain (i.e., zinc finger) and a cleavage domain (i.e., nuclease). Zinc finger binding domains may be engineered to recognize and bind to any nucleic acid sequence of choice. The cleavage domain portion of the ZFNs may be obtained from any suitable nuclease or exonuclease such as restriction nucleases and homing nucleases. See, for example, Belfort & Roberts, 1997, Homing nucleases: keeping the house in order, Nucleic Acids Res 25(17):3379-3388, incorporated by reference. A cleavage domain may be derived from an enzyme that requires dimerization for cleavage activity. Two ZFNs may be required for cleavage, as each nuclease comprises a monomer of the active enzyme dimer. Alternatively, a single ZFN may comprise both monomers to create an active enzyme dimer. Restriction nucleases present may be capable of sequence-specific binding and cleavage of DNA at or near the site of binding. Certain restriction enzymes (e.g., Type IIS) cleave DNA at sites removed from the recognition site and have separable binding and cleavage domains. For example, the Type IIS enzyme FokI, active as a dimer, catalyzes double-stranded cleavage of DNA, at 9 nucleotides from its recognition site on one strand and 13 nucleotides from its recognition site on the other. The FokI enzyme used in a ZFN may be considered a cleavage monomer. Thus, for targeted double-stranded cleavage using a FokI cleavage domain, two ZFNs, each comprising a FokI cleavage monomer, may be used to reconstitute an active enzyme dimer. See Wah, et al., 1998, Structure of FokI has implications for DNA cleavage, PNAS 95:10564-10569; U.S. Pat. Nos. 5,356,802; 5,436,150; 5,487,994; U.S. Pub. 2005/0064474; U.S. Pub. 2006/0188987; and U.S. Pub. 2008/0131962, each incorporated by reference.

Transcription activator-like effector nucleases (TALENs) cut genetic material in a sequence-specific matter and can be designed, or programmed, to target specific viral targets. TALENs contain the FokI nuclease domain at their carboxyl termini and a class of DNA binding domains known as transcription activator-like effectors (TALEs). TALEs are composed of tandem arrays of 33-35 amino acid repeats, each of which recognizes a single base-pair in the major groove of target viral DNA. The nucleotide specificity of a domain comes from the two amino acids at positions 12 and 13 where Asn-Asn, Asn-Ile, His-Asp and Asn-Gly recognize guanine, adenine, cytosine and thymine, respectively. That pattern allows one to program TALENs to target viral nucleic acid. TALENs use a nonspecific DNA-cleaving nuclease fused to a DNA-binding domain that can be to target essentially any sequence. For TALEN technology, target sites are identified and expression vectors (e.g., plasmids) for the TALENs may be made, or the TALENs are ordered as proteins. Linearized expression vectors (e.g., by NotI) may be used as template for mRNA synthesis. A commercially available kit may be use such as the mMESSAGE mMACHINE SP6 transcription kit from Life Technologies (Carlsbad, Calif.). See Joung & Sander, 2013, TALENs: a widely applicable technology for targeted genome editing, Nat Rev Mol Cell Bio 14:49-55, incorporated by reference.

In general, the CRISPR terminology refers to gene editing systems that were observed as RNA-guided nucleases found as part of bacterial immune systems.

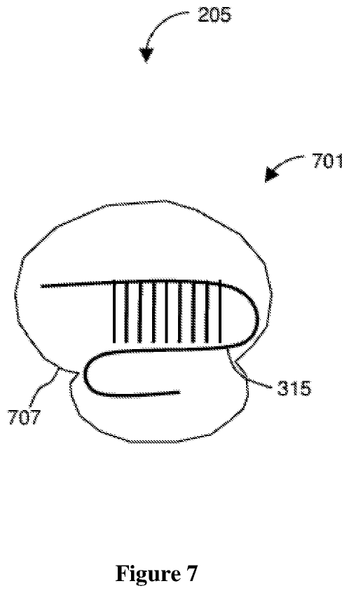

FIG. 7 shows one embodiment of gene editing reagents 205 that includes protein, specifically where a Cas endonuclease 707 is complexed with a guide RNA 315 that includes an approximately 20 base targeting sequence preferably complementary to a target in a globin gene cluster. The Cas endonuclease 707 and guide RNA 315 are complexed together as a ribonucleoprotein (RNP) 701. Accordingly, the gene editing reagents 205 in a composition or method of the disclosure may include at least one Cas endonuclease 707 (or a nucleic acid encoding the Cas endonuclease such as a DNA plasmid, other expression vector, or mRNA).

Embodiments of the invention use proteins that are originally encoded by genes that are natively associated with clustered regularly interspaced short palindromic repeats (CRISPR) in bacterial genomes. Preferred embodiments use a CRISPR-associated (Cas) endonuclease. For such embodiments, the gene editing reagents include a protein such as a Cas endonuclease complexed (to form a complex) with a guide RNA that targets the Cas endonuclease to a specific sequence. That complex is a ribonucleoprotein (RNP). Any suitable Cas endonuclease or homolog thereof may be used. A Cas endonuclease (catalytically active or deactivated) may be Cas9 (e.g., spCas9), catalytically inactive Cas (dCas such as dCas9), Cpf1 (aka Cas12a), C2c2, Cas13, Cas13a, Cas13b, e.g., PsmCas13b, LbaCas13a, LwaCas13a, AsCas12a, others, modified variants thereof, and similar proteins or macromolecular complexes.

The host bacteria capture small DNA fragments (˜20 bp) from invading viruses and insert those sequences (termed protospacers) into their own genome to form a CRISPR. Those CRISPR regions are transcribed as pre-CRISPR RNA (pre-crRNA) and processed to give rise to target-specific crRNA. Invariable target-independent trans-activating crRNA (tracrRNA) is also transcribed from the locus and contributes to the processing of precrRNA. The crRNA and tracrRNA have been shown to be combinable into a single guide RNA. As used herein, “guide RNA” or gRNA refers to either format. The gRNA forms a RNP with Cas9, and the RNP cleaves a target that includes a portion complementary to the guide sequence in the gRNA and a sequence known as protospacer adjacent motif (PAM). The RNA-guided nucleases are programmed to target a specific viral nucleic acid by providing a gRNA that includes a ˜20-bp guide sequences that is substantially complementary to a target in viral nucleic acid. The targetable sequences include, among others, 5″-X 20NGG-3″ or 5″-X 20NAG-3″; where X 20 corresponds to the 20-bp crRNA sequence and NGG and NAG are PAMs. It will be appreciated that recognition sequences with lengths other than 20 bp and PAMs other than NGG and NAG are known and are included within the scope of the invention.

Argonaute proteins are a family of proteins that play a role in RNA silencing as a component of the RNA-induced silencing complex (RISC). The Argonaute of the archaeon Pyrococcus furiosus (PfAgo) uses small 5′-phosphorylated DNA guides to cleave both single stranded and double stranded DNA targets, and does not use RNA as guide or target.

NgAgo uses 5′ phosphorylated DNA guides (so called “gDNAs”) and appear to exhibit little preference for any certain guide sequences and thus may offer a general-purpose DNA-guided programmable nuclease. NgAgo does not require a PAM sequence, which contributes to flexibility in choosing a genomic target. NgAgo also appears to outperform Cas9 in GC-rich regions. NgAgo is only 887 amino acids in length. NgAgo randomly removes 1-20 nucleotides from the cleavage site specified by the gDNA. Thus, PfAgo and NgAgo represent potential DNA-guided programmable nucleases that may be modified for use as a composition of the invention.

In any of the compositions and methods of the disclosure, the gene editing reagents may be included in any suitable format including any of protein, messenger RNA, DNA, RNP, or a combination thereof. For example, RNPs may be delivered into cells by electroporation, chemical poration, or via liposomal mediated delivery. The gene editing reagents may be delivered in a DNA sense (e.g., as a plasmid or in a viral vector) for transcription and translation into active proteins in the target cells. In some embodiments, the gene editing reagents 205 are delivered as nucleic acid, e.g., the Cas endonuclease, and are packaged with a globin gene using one or more lentiviral or adeno-associated virus (AAV) vector. The globin gene may be included as a segment of DNA that also includes one or more of a promoter, a fluorescent protein, an SV40 sequence, and a poly(A) sequence. The gene and/or the reagents may be delivered as a plasmid or other similar vector.

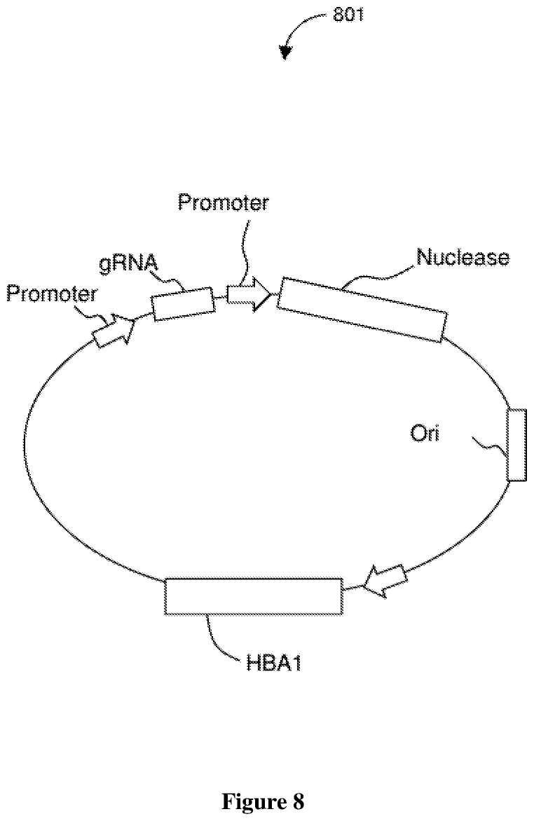

FIG. 8 shows a plasmid 801 for treating alpha thalassemia by gene editing. In the depicted embodiment, the gene editing reagents 205 are in a DNA form, specifically, with a Cas endonuclease in a DNA plasmid 801. The plasmid 801 in this case also encodes the guide RNA and includes an alpha-globin gene (here, HBA1). Each of the depicted elements may optionally be included on one plasmid or distributed across one or more. As shown, the plasmid 801 is unique because it includes a segment that encodes a guide RNA 315 that includes a targeting portion that is complementary to a target 301 within a globin gene cluster (because that segment of the plasmid 801 is transcribed into the gRNA 315, that segment is antisense to the gRNA 315; but because the gRNA 315 is antisense to the target in the globin gene cluster, the plasmid 801 itself includes, within the gRNA segment, a 20 base segment that matches an identified 20 base segment in a human globin gene locus that is adjacent a PAM). Other embodiments are within the scope of the disclosure. For example, the gene editing reagents may be delivered to the cells or fetus in an mRNA format.

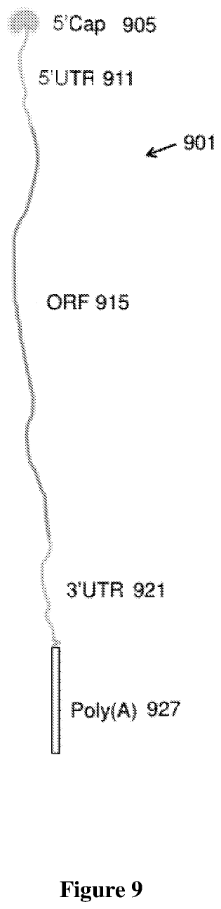

FIG. 9 shows an mRNA 901 with an open reading frame 915 that can be translated into a gene editing protein. The mRNA also includes a 5′ cap 805, a 5′ untranslated region 911, a 3′ untranslated region 921, and a poly-A tail 927. A practitioner may select to use gene editing reagents 205 in a protein, RNP, DNA, or mRNA format dependent on a desired persistence or stability in the target cells. For example, protein or RNP will generally be cleared via proteases according to a predictable time course, and the quantity of protein or RNP will never be amplified after delivery. On the one hand, a format that relies on the delivery of protein is thus useful where it is intended to limit or control a total quantity of reagent that is active in the cells and a duration of activity. On the other hand, DNA can persist: it can be delivered in a viral expression vector, it can integrate into a host genome, a plasmid may include an origin of replication (Ori), and the DNA can be transcribed into multiple mRNAs that may be translated into many more proteins. The mRNA format may be chosen as a middle ground; a quantity can be amplified (relative to delivery of protein) by using the endogenous translation machinery. The mRNA and DNA formats may offer attractively compact delivery formats for compositions of the disclosure. The mRNA and DNA formats may avoid unwanted immune response consequences. Using the mRNA 901, it is possible to treat a patient with a composition that includes a globin gene as DNA and the gene editing reagents in a format that includes at least one mRNA that, when is introduced into an fetus or cell, is translated into a gene editing nuclease by a ribosome.

Thus, the gene editing reagents may include a transcription activator like effector nuclease (TALEN), a zinc-finger nuclease (ZFN), a Cas endonuclease—or nucleic acid encoding the TALEN, ZFN, or Cas endonuclease—wherein the TALEN, ZFN, or Cas endonuclease is designed to introduce a globin gene into a target locus. That designed property of target-specificity of the gene editing reagent makes each molecule unique for its purpose in that at a least a portion of the molecule is designed to be cognate to the target locus. Preferred target loci may include an alpha-globin gene cluster in chromosome 16, a beta-globin cluster, or a genomic safe harbor (e.g., a safe harbor such as AAVS1, CCR5, or hROSA26). The gene editing reagents 205 may be included as DNA that is transcribed after the composition is introduced into a fetus or cell, as mRNA or as a protein or RNP. Whichever format is used (DNA, mRNA, protein), a suitable packaging vector or particle may be used. The gene editing reagents (such as ZFNs, TALENs, or CRISPR) could be delivered using viral vectors such as AAV vectors (with numerous capsid proteins, including but not limited to aav6, or others that target HSCs or relevant progeny populations to edit RBCs), lentiviral vectors, or other retroviral vectors.

### IV. Compositions for Gene Replacement

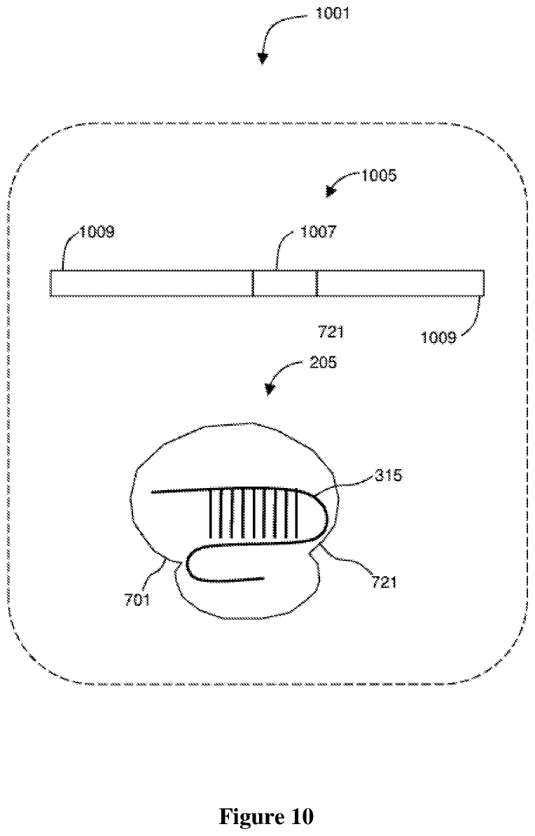

FIG. 10 shows a composition 1001 for treating ATM via gene replacement. The composition 1001 that includes a globin gene 1007 and gene editing reagents 205 that, when the composition 1001 is introduced in vivo into the fetus or ex vivo into HSCs derived from the fetus or the patient after birth, insert the globin gene 1007 into genomic material. In the depicted embodiment, the composition 1001 includes a segment of DNA 1005 that includes the globin gene 1007 (here, an alpha-globin gene such as HBA1). The segment of DNA 1005 may also include one or more of a promoter, a fluorescent protein, an SV40 sequence, and a poly(A) sequence.

The gene editing reagents 205 include a Cas endonuclease 707 complexed with a guide RNA 315 that includes an approximately 20 base targeting sequence preferably complementary to a target in a globin gene cluster. As known in the art, the targeting sequence of gRNA does not need to be perfectly complementary as the system tolerates some mismatches. Preferably the targeting sequence is at least about 75% complementary, more preferably at least about 90%. The Cas endonuclease 707 and guide RNA 315 are complexed together as a ribonucleoprotein (RNP) 701. The depicted gRNA 315 has an approximately 20 base targeting segment that is substantially or perfectly complementary to the target in the globin gene cluster, where the target is an identified 20 base segment in a human globin gene locus that is adjacent a PAM for the Cas endonuclease 707.

In a preferred embodiment, the segment of DNA 1005 includes ends 1009 that substantially match (e.g., over at least a few dozen to a few hundred bases) homologous segments in the globin gene cluster. Note that the composition 1001 is defined and given its unique properties by one or any combination of: the globin gene (e.g., HBA1) in the segment of DNA 1005, the ends 1009 that substantially match homologous segments in the globin gene cluster, and the approximately 20 base targeting segment (in the gRNA 315) that is complementary to the target in the globin gene cluster. The ends 1009 that substantially match homologous segments in the globin gene cluster promote integration of the HBA1 gene into genomic material in the cell (e.g., within the short arm of chromosome 16) by homology-directed repair (HDR) using the cell's native HDR system. Thus the depicted composition 1001 is suitable for treatment of alpha thalassemia. Preferably, the globin gene 1007 is an alpha-globin gene and the gene editing reagent 205 comprise a first Cas9 ribonucleoprotein (RNP) 701 that includes a first guide RNA (g RNA) 315 and a second Cas9 RNP, wherein the first Cas9 RNP and the second Cas9 RNP bind to a locus within an alpha-globin gene cluster in chromosome 16 of the genomic material, and introduces the alpha-globin gene into the locus within the alpha-globin gene cluster.

The composition 1001 is useful in methods 101, 501 for treating alpha thalassemia by gene replacement/gene insertion. Other embodiments are within the scope of the disclosure. In some embodiments, the methods 101, 501 are used with composition for gene editing to treat alpha thalassemia.

### V. Compositions for Gene Editing

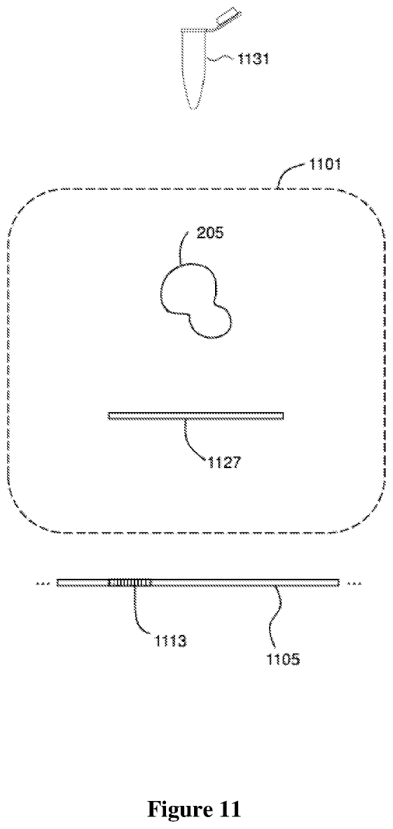

FIG. 11 shows a composition 1101 useful for editing a globin gene 1113 within a genome 1105. The composition 1101 preferably includes gene editing reagents 205 and optionally includes a replacement segment 1127. The gene editing reagents 205 have properties discussed above and include elements shown elsewhere herein. The reagents 205 may include DNA, mRNA, protein, or RNP, and may provide a TALEN, ZFN, or Cas endonuclease that targets a predetermined target in a globin gene 1113. The composition 1101 (or any other composition of the disclosure may be provided in any suitable package or container such as a vial or tube 1131 such as the microcentrifuge tube sold under the trademark EPPENDORF. The composition may include, or be provided in a kit that also includes, blood comprising alpha globin for transfusion to the fetus. The gene 1113 or its operon or its promoters or any other regulatory segment may be targeted. For example, the composition 1101 may be used to insert a promotor (e.g., in the replacement segment 1127) near a gene. Or the composition 1101 may be used to knock-out a gene (e.g., introduce a stop codon). The gene editing reagents 205 are preferably targeted to a gene in the alpha-globin cluster are useful for treating alpha thalassemia according to a suitable strategy.

One suitable strategy for treating alpha thalassemia may include increasing the production or persistence of zeta globin. The composition 1101 may mutate/delete a repressor by which zeta-globin is naturally silenced at the end of the first trimester so that zeta globin is expressed into the second trimester. Or the composition may introduce a mutation into a 3′ translated region of a zeta-globin gene, which mutation leads to mRNA zeta-globin transcripts that are more stable than for un-mutated versions. Thus, some embodiments of the composition 1101 are designed such that—when the composition is introduced in vivo into the fetus or patient or ex vivo into HSCs derived from the fetus or the patient after birth—the zeta-globin gene is transcribed into zeta-globin transcripts that are more stable than transcripts from a wild-type zeta-globin gene that is the same as the zeta-globin gene but that does not have the mutation in the 3′ sequence of the translated region. The composition 1101 may be delivered into cells (HSCs, RBCs, progeny, or precursors thereof) obtained from the fetus and those cells or the composition 1101 itself may injected into the fetus, either into the bloodstream or into the umbilical cord or placenta or fetal liver or heart, such that the fetus expresses the globin gene for at least a trimester.

Thus the composition 1101 is useful for treatment of alpha thalassemia and includes gene editing reagents 205 that when introduced into a fetus or a hematopoietic stem cell (HSC) introduce a change into a sequence within a globin gene within genomic material of the fetus or the HSC. The composition 1101 may be provided in a viral vector or non-viral particle. The viral vector or the non-viral particle optionally includes one or more targeting molecules that target the viral vector or the non-viral particle to the target cells. As shown, the gene editing reagents 205 include a Cas endonuclease and one or more guide RNAs that target the globin gene. However, the composition 1101 could include nucleic acid encoding the Case endonuclease (e.g., a plasmid, a viral expression vector, or mRNA). In preferred embodiments, the gene editing reagents are designed with sequences cognate to a zeta-globin gene such that the gene editing reagents 205 introduce a mutation into the zeta-globin gene 1113.

The gene editing reagents 205 may be used to introduce a mutation into a repressor region in a zeta-globin gene 1113. The repressor region includes a NF-kB binding region in a 3′ end of the zeta-globin gene. By mutating it, the binding of NF-kB is prevented, and zeta-globin is not silenced. After the change is introduced within the sequence of the globin gene 1113 within the fetus, the zeta-globin gene is not silenced at the end of a first trimester, and the zeta globin is expressed during a second trimester.

The gene editing reagents 205 may be used to introduce a mutation into a 3′ sequence of a translated region of a zeta-globin gene 1113. After the change is introduced within the sequence of the globin gene, the mutated zeta-globin gene is transcribed into modified transcripts, wherein the modified transcripts persist for longer than similar but unmodified transcripts.

The composition 1101 is useful in an ex vivo method of treating alpha thalassemia that includes obtaining a sample comprising cells from a fetus. The cells preferably include fetal HSCs, or RBCs, or precursors of either. The composition 1101 is used to modify a zeta-globin gene 1113 within the cells. Preferably, an assay is performed to validate that the cells or their progeny express the modified zeta-globin gene (e.g., an ELISA test for zeta globin). The method may include surgically accessing the fetus in the pregnant woman and introducing the cells or their progeny into fetal circulation by injection into the fetus, either into the bloodstream or into the fetal liver or heart or into the placenta (see FIG. 6), whereby the modified zeta-globin gene in the fetus is resistant to gene silencing and is expressed persistently.

### VI. Particles and Vectors for Delivery

Gene editing reagents 205 of the disclosure may include a transcription activator like effector nuclease (TALEN), a zinc-finger nuclease (ZFN), a Cas endonuclease—or nucleic acid encoding the TALEN, ZFN, or Cas endonuclease—wherein the TALEN, ZFN, or Cas endonuclease is designed to introduce a globin gene into a target locus. That designed property of target-specificity of the gene editing reagent makes each molecule unique for its purpose in that at a least a portion of the molecule is designed to be cognate to the target locus. Preferred target loci may include an alpha-globin gene cluster in chromosome 16, a beta-globin cluster, or a genomic safe harbor (e.g., a safe harbor such as AAVS1, CCR5, or hROSA26). The gene editing reagents 205 may be included as DNA that is transcribed after the composition is introduced into an fetus or cell, as mRNA or as a protein or RNP. Whichever format is used (DNA, mRNA, protein), a suitable packaging vector or particle may be used.

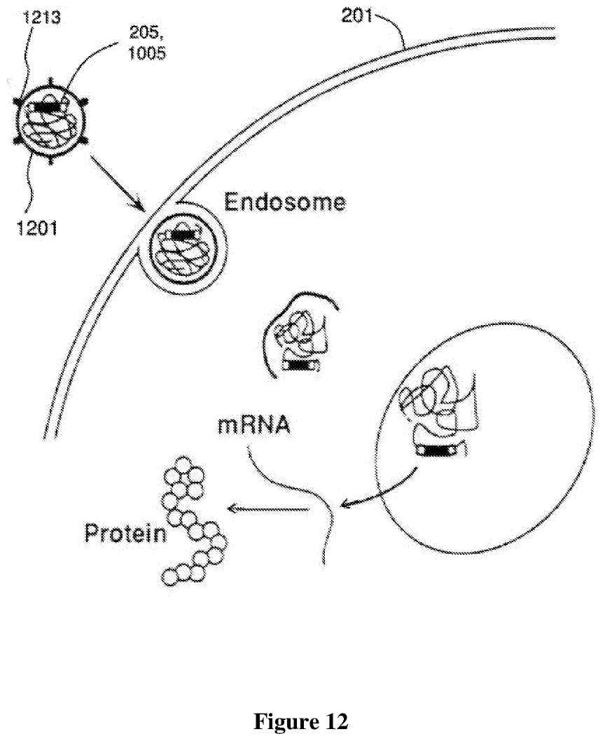

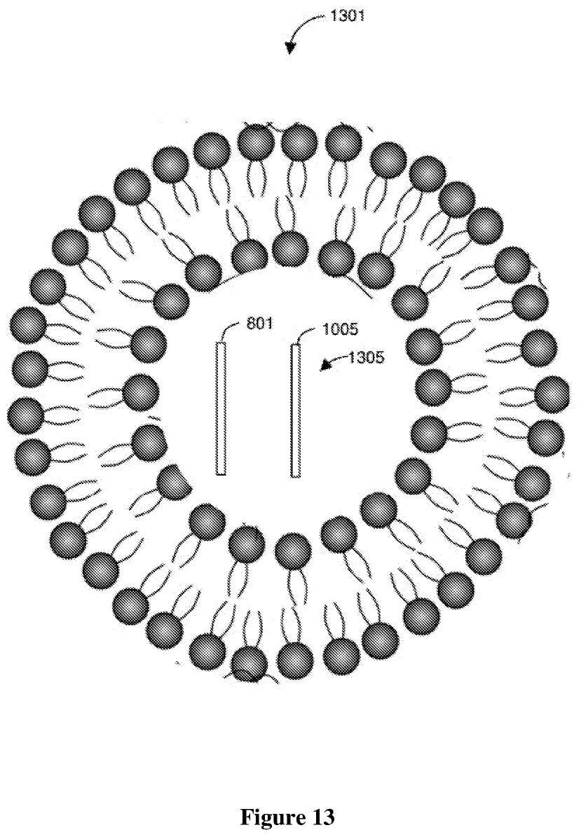

FIG. 12 shows a viral vector 1201 such as an adeno-associated viral vector. The viral vector 1201 may include gene editing reagents 205, a segment of DNA 1005 that includes the globin gene, or both. The viral vector may include a surface modification 1213, such as an antibody, that targets the vector to a cell 201, such as a fetal HSC. The surface modification 1213 may be an antibody linked to an external surface of the viral delivery vector, e.g., for targeting hematopoietic stem cells, or precursors thereof.

A composition of the disclosure may also be packaged or delivered using a non-vector.

Any suitable particles may be included. The particle may be a solid lipid nanoparticle. Additionally or alternatively, liposomes may be used to deliver composition due to multiple cationic surface groups, which interact with anionic nucleic acids and form lipoplexes.

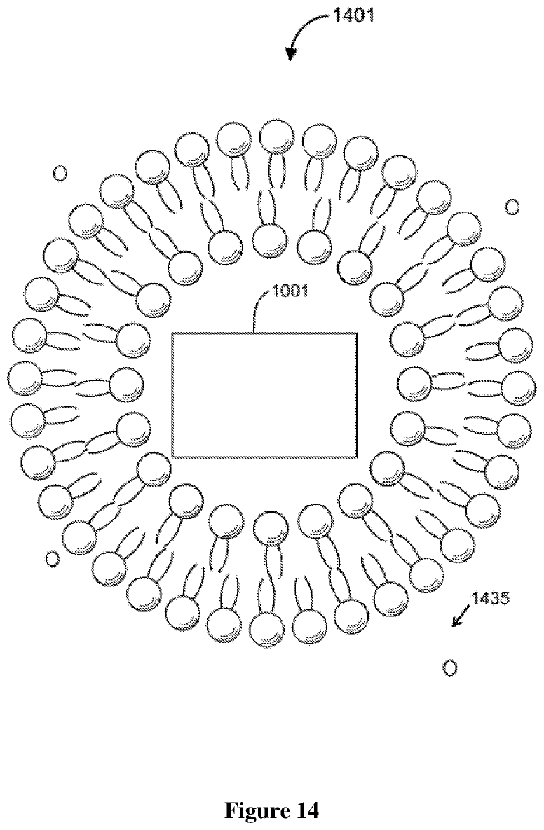

FIG. 13 shows a liposome 1301 for delivery of a composition of the disclosure. The liposome 1301 preferably includes the gene editing reagents 205 (here shown as a plasmid 801) and optionally any DNA segment 1005 that includes a globin gene. In the depicted embodiment, the plasmid 801 includes a segment that encodes a gRNA 315. Where the liposome 1301 packages nucleic acids 1305, those nucleic acids 1305 may include one or any combination of a plasmid 801, a guide RNA 315, a segment of DNA 1005 including a globin gene, a replacement segment 1127, or an mRNA 901.

A composition of the disclosure may include a plurality of the liposomes 1301 that, collectively, envelope one or more of a globin gene and/or the gene editing reagents 205 (e.g., as a plasmid 801). For example, the DNA segment 1005 may a zeta-globin gene that includes, e.g., a mutation in a repressor region (such as an NF-kB binding region in a 3′ portion of the zeta-globin gene), or a mutation in a 3′ sequence of a translated region. In the depicted embodiment, delivery of the liposomes 1301 to cells (HSCs, RBS, or progeny or precursors) in a fetus, causes those cells to stably express zeta globin as they mature and circulate in the fetus, because the gene is not silenced and/or the transcripts are stabilized by the indicated mutations. The liposomes 1301 could similarly be used in the method 1001 to introduce an alpha-globin gene into a genome of cells (HSCs, RBS, or progeny or precursors) in fetus or that are delivered into the fetus, to cause those cells to express alpha globin to treat alpha thalassemia.

Either a solid lipid nanoparticle or a liposome preferably includes at least one cationic lipid. Encapsulating the composition in a plurality of nanoparticles comprising a cationic lipid may proceed by any suitable method. Methods for preparation may include direct mixing between cationic liposomes and nucleic acids 1305 in solution, or rehydration of a thin-layer lipid membrane with nucleic acids 1305 in solution. The dispersion of cationic lipid/nucleic acids 1305 in the aqueous solution often results in heterogeneous complexes, sometimes still referred to as cationic liposomes, but more accurately called lipoplexes. Lipoplexes can encapsulate nucleic acid cargos up to 90% of the input dose. See Wang, 2015, Delivery of oligonucleotides with lipid nanoparticles, Adv Drug Deliv Rev 87:68-80, incorporated by reference.

In some embodiments, nucleic acids 1305 interact electrostatically with a preformed DOTAP (1,2-dioleoyl-3-trimethylammonium-propane)/cholesterol (1:1 molar ratio) liposome 1301. Electrostatic interaction between the cationic lipid head group and the backbone of nucleic acids 1305 drives encapsulation of nucleic acids 1305 in cationic liposomes. This yields a self-assembly, liposome-based, core membrane nanoparticle formulation. The electrostatic interaction promotes the self-assembly by inducing lipid bilayers to collapse on the core structure, resulting in spherical, solid, liposomal nanoparticles with a core/membrane structure. See Wang, 2013, Systematic delivery of modified mRNA encoding herpes simplex virus 1 thymidine kinase for targeted cancer gene therapy, Mol Ther 21(2):358-367, incorporated by reference.

Methods for preparation may include direct mixing between cationic liposomes and nucleic acids 1305 in solution, or rehydration of a thin-layer lipid membrane with RNA in solution. The dispersion of cationic lipid/nucleic acids 1305 complexes in the aqueous solution may result in heterogeneous complexes, sometimes still referred to as cationic liposomes, aka lipoplexes. Lipoplexes can encapsulate nucleic acid cargos up to 90% of the input dose. See Wang, 2015, Delivery of oligonucleotides with lipid nanoparticles, Adv Drug Deliv Rev 87:68-80, incorporated by reference.

Generally, cationic lipids are classified into three major categories based on the head group structure: monovalent lipids such as N (1-(2,3-dioleyloxy) propyl)-N,N,N-trimethylammonium chloride (DOTMA) and 1,2-dioleyl-3-trimethylammonium-propane (DOTAP); multivalent lipids such as dioctadecylamidoglycylspermine (DOGS); and cationic lipid derivatives such as 36-(N—(N′,N′-dimethylaminoethane)-carbamoyl) cholesterol (DC-Chol). The hydrophobic chains provide the nanoparticle with different features. It may be found that the myristoyl (C14) chain is optimal for transfection compared to C16 and C18 chains. Longer chains increase the phase transition temperature and reduce the fluidity of the lipid membrane, which may be unfavorable for lipid membrane fusion. Similarly, unsaturated alkyl chains with considerably higher lipid fluidity may lead to a higher transfection efficiency compared to saturated alkyl chain lipids

Cationic lipids may be used as vectors to condense and deliver anionic nucleic acids through electrostatic interactions. By modulating the ratio of cationic lipids and nucleic acids, the excess cationic coating may aid binding of vectors with negatively charged cell surfaces and the endosomal membrane to help cytoplasmic delivery of nucleic acids. Electrostatic interaction between the cationic lipid head group and the backbone of nucleic acids drives encapsulation of nucleic acids 1305 in cationic liposomes. Optionally, the nanoparticles 105 are PEG-ylated.

FIG. 14 is a cartoon of a lipid nanoparticle (LNP) 1401 according to certain embodiments. The LNP 1401 may include any composition of the disclosure such as, for example, the composition 1001. LNPs may be about 100-200 nm in size and may optionally include a surface coating of a neutral polymer such as PEG to minimize protein binding and unwanted uptake. The nanoparticles 1401 are optionally carried by a carrier 1435, such as water, an aqueous solution, suspension, or a gel. E.g., LNPs may be included in a formulation or preparation for topical delivery such as a suspension or gel. Such as a formulation may include chemical enhancers, such as fatty acids, surfactants, esters, alcohols, polyalcohols, pyrrolidones, amines, amides, sulfoxides, terpenes, alkanes and phospholipids.

Use of an LNP may enhance the solubility of the payload, provide sustained and controlled release, and deliver higher concentrations of payload to target areas due to an Enhanced Permeation and Retention (EPR) effect. Lipid-based nanoparticles (liposomes and solid-lipid nanoparticles) may be used.

In certain embodiments, LNPs are suspended in a buffer. The buffer may include a penetration enhancing agent such as sodium lauryl sulfate (SLS). SLS is an anionic surfactant that enhances penetration into the skin by increasing the fluidity of epidermal lipids. The increase in lipid fluidity below the applied site may allow SLS to diffuse optimally. SLS could thus increase intra-epidermal drug delivery without increasing transdermal delivery. Methods may include use of a buffer such as a pH=6 200 mM phosphate buffer, optionally with SLS at about 1 to 10% wt/wt, i.e., about 35 to 250 mM SLS.

Lipid nanoparticles optionally may be delivered via a gel, such as a polyoxyethylene-polyoxypropylene block copolymer gel (optionally with SLS). Poloxamers are nonionic triblock copolymers composed of a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exist that have slightly different properties. For the generic term “poloxamer”, these copolymers are commonly named with the letter “P” (for poloxamer) followed by three digits: the first two digits×100 give the approximate molecular mass of the polyoxypropylene core, and the last digit×10 gives the percentage polyoxyethylene content (e.g., P407=poloxamer with a polyoxypropylene molecular mass of 4,000 g/mol and a 70% polyoxyethylene content). For the Pluronic and Synperonic tradenames, coding of these copolymers starts with a letter to define its physical form at room temperature (L=liquid, P=paste, F=flake (solid)) followed by two or three digits, The first digit (two digits in a three-digit number) in the numerical designation, multiplied by 300, indicates the approximate molecular weight of the hydrophobe; and the last digit×10 gives the percentage polyoxyethylene content (e.g., L61 indicates a polyoxypropylene molecular mass of 1,800 g/mol and a 10% polyoxyethylene content).

Lipid nanoparticles may be freeze-dried (e.g., using dextrose (5% w/v) as a lyoprotectant). LNPs may be held in an aqueous suspension or in an emulsification, e.g., with lecithin. may be encapsulated in LNPs using a self-assembly process. LNPs are prepared using ionizable lipid L319, distearoylphosphatidylcholine (DSPC), cholesterol and PEG-DMG at a molar ratio of 55:10:32.5:2.5 (L319:DSPC:cholesterol:PEG-DMG). The payload is introduced at a total lipid to payload weight ratio of ˜10:1. A spontaneous vesicle formation process is used to prepare the LNPs. Payload is diluted to ˜1 mg/ml in 10 mmol/l citrate buffer, pH 4. The lipids are solubilized and mixed in the appropriate ratios in ethanol. Syringe pumps are used to deliver the payload solution and lipid solution at 15 and 5 ml/min, respectively. The syringes containing payload solution and lipid solution are connected to a union connector (0.05 in thru hole, #P-728; IDEX Health & Science, Oak Harbor, Wash.) with PEEK high-performance liquid chromatography tubing (0.02 in ID for siRNA solution and 0.01 in ID for lipid solution). A length of PEEK high-performance liquid chromatography tubing (0.04 in ID) is connected to the outlet of the union connector and led to a collection tube. The ethanol is then removed and the external buffer replaced with phosphate-buffered saline (155 mmol/l NaCl, 3 mmol/l Na2HPO4, 1 mmol/l KH2PO4, pH 7.5) by either dialysis or tangential flow diafiltration. Finally, the LNPs are filtered through a 0.2 μm sterile filter. LNPs preferably contain an ionizable cationic lipid/phosphatidylcholine/cholesterol/PEG-lipid (50:10:38.5:1.5 mol/mol), encapsulated payload-to-total lipid ratio of ˜0.05 (wt/wt) and a diameter of ˜80 nm. Payload-LNP formulations may be stored at −80° C. at a concentration of mRNA of ˜1 μg/μl. See Maier, 2013, Biodegradable lipids enabling rapidly eliminating lipid nanoparticles for systemic delivery of RNAi therapeutics, Mol Ther 21(8):1570-1578, incorporated by reference. For background see, WO 2016/089433 A1, incorporated by reference.

Whichever particle/vector is used, preferably the particle or vector includes a globin gene and/or the gene editing reagents 201. Compositions of the disclosure may include a plurality of lipid nanoparticles having the globin gene and the gene editing reagents embedded therein. For example, the plurality of lipid nanoparticles comprises at least: a first solid lipid nanoparticle comprising a segment of DNA that includes the globin gene; a second solid lipid nanoparticle that includes at least one Cas endonuclease complexed with a guide RNA (gRNA) that targets the Cas endonuclease to a locus within an alpha-globin gene cluster in chromosome 16.

### VII. Kits

Compositions of the disclosure may be packaged as or provided in kits.

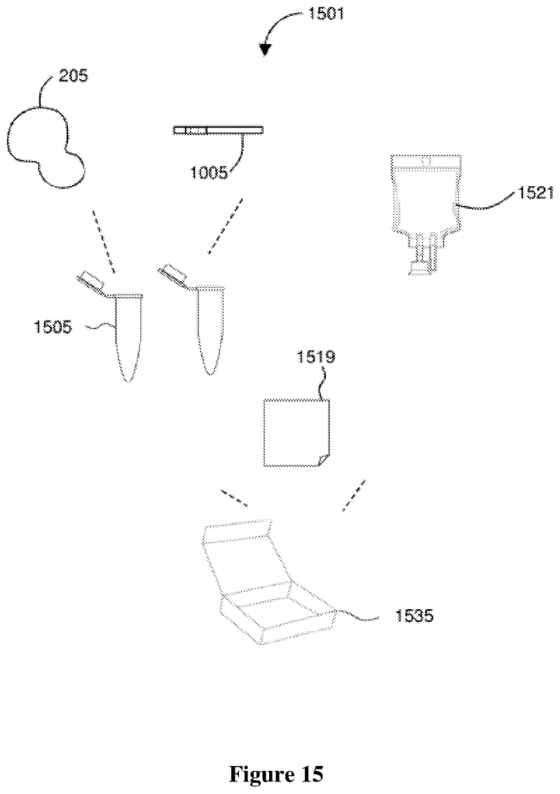

FIG. 15 shows a kit 1501 as may be provided with any embodiment of the disclosure. The kit 1501 preferably includes gene editing reagents 205 and any DNA segment 1005 that is used in gene editing, optionally within a one or more suitable containers 1505 such as a well of a multi-well plate, test tube, or micro-centrifuge tube such as the tube sold under the trademark EPPENDORF by Fisher Scientific Co. L.L.C. (Pittsburgh, Pa.). Elements of the kit may optionally be shipped or received together in a package 1535 and may optionally include instructions 1519 or other supplementary material. The kit 1501 may further include a blood bag 1521 (e.g., containing healthy blood). The kit 1501 may include additional reagents that promote integration of the globin gene into the genomic material, wherein the additional reagents include one or more of a polymerase, a ligase, dNTPs, a co-factor, and a topoisomerase.

One significant feature of the kit 1501 is that it provides a convenient format for the inclusion of elements that support embodiments of the methods herein. For example, an important insight of the invention is that a beneficial approach to treating ATM via gene editing or gene replacement is that the gene editing reagents may be co-delivered with a blood transfusion as has previously been delivered to fetuses suffering from alpha thalassemia. The blood transfusion includes blood that includes a globin such as alpha globin. Thus, treating an ATM patient may involve obtaining a preparing a kit 1501 that includes gene editing reagents 205 and a blood bag 1521 with blood to be transfused into the patient.

In some embodiments, the kit includes one or more surgical tools for delivering the globin gene and the gene editing reagents into the circulation of the fetus. The kit 1501 may include a spinal needle, tubing, syringes, or other tools.

### VIII. Other Strategies

Embodiments of methods and compositions of the disclosure may be used in strategies focusing on increasing the production of zeta globin to make up for the absence of alpha globin.

During embryonic development, the zeta-globin gene, which is 5′ of the alpha-globin genes, first contributes to embryonic Hb.

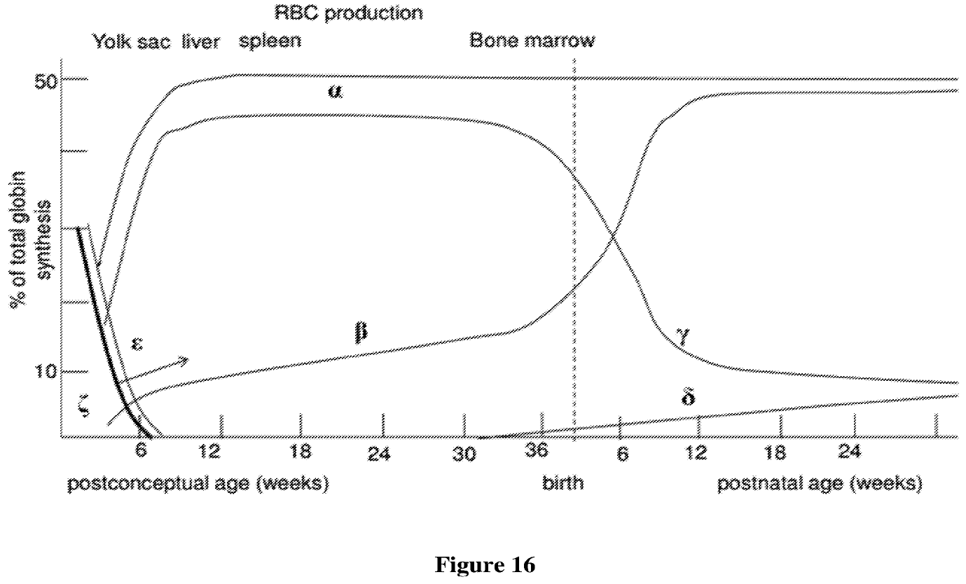

FIG. 16 shows the genes of the alpha and beta chains, and the how each globin chain contributes to the formation of embryonic, fetal, and adult hemoglobin. As shown, the zeta globin is expressed early, and naturally is expressed up until about 6 weeks post-conception. The zeta gene then gets silenced, and alpha globin starts to get produced during fetal liver hematopoiesis. In the figure, the arrow drawn on the zeta globin profile indicates that methods and compositions of the disclosure may be useful to shift the profile of zeta-globin expression to the right, i.e., to increase persistence or expression over time.

Silencing of zeta-globin is both due to decreased transcription of the gene (due to silencers that are located in cis (such as at the 3′ region) as well as due to decreased translation. There may be un-identified silencers that act in trans. The zeta mRNA has decreased stability compared to alpha globin due to sequences in its 3′ region, leading to lower levels of zeta chain protein production. For background, see Russell, 1998, Sequence divergence in the 3′ untranslated regions of human zeta- and alpha-globin mRNAs mediates a difference in their stabilities and contributes to efficient alpha-to-zeta gene development switching, Mol Cell Biol 18:2173-83, incorporated by reference.

Human fetuses with ATM survive the embryonic development due to the production of zeta globin; they become hypoxic in the second trimester when the switch from zeta to alpha globin takes place. Therefore, improving the production or persistence of zeta globin could treat patients with ATM. Since zeta globin is produced by fetuses with ATM, the presence of fetal RBCs with zeta globin in maternal blood could also be developed as a prenatal non-invasive diagnostic test for this disease.

Detection of zeta globin in adult blood could be a blood test to determine who is a carrier for the mutation in couples at risk. For background, see Tang, 1992, Blood 80:517-22, incorporated by reference. Regulation of zeta globin was studied in the 1990s using molecular methods available at the time and there are several insights that can be extrapolated to a medical therapy, none of which have been reduced to practice. One important insight is that there is a putative repressor region at the 3′ end of the zeta gene that binds to NFkB, and introducing a 2 bp mutation in a plasmid expressing this gene allows production of intact zeta globin.

Our understanding of human globin genes is useful to develop novel approaches to block their silencing in utero. For background, see Wang 1999, Embo J 18:2218, incorporated by reference. Therefore, this disclosure provides compositions and methods that employ a gene editing strategy that introduces a mutation in the endogenous zeta gene to treat a host with alpha thalassemia. The indicated strategies are useful to increase the amount of zeta globin to make up for the absence of alpha globin.

Compositions and methods of the disclosure may be used for silencing the repressor region. Some strategies to achieve this are to mutate the known repressor region (NFkB-binding region in the 3′ end of the zeta gene)3, using tools such as gene editing reagents, including but not limited to TALENs, ZFNs, CRISPR/Cas, or base editors. Compositions and methods of the disclosure may be used for increasing the stability of the zeta-globin mRNA by disrupting the 3′ sequence of the translated region to allow it to bind to the mRNA stabilizing complex. Compositions and methods of the disclosure may be used for introducing additional copies of the zeta-globin gene that is appropriately edited to disallow the usual silencing, such as by mutating the 3′ end that binds to NFkB, or modifying additional relevant sites that are in cis or in trans to the zeta-globin gene. The inserted zeta-globin gene may be under the control of the alpha- or beta-locus control region or similar promoter to ensure appropriate transcription. Compositions and methods of the disclosure may be used for activating an enhancer of zeta-globin. The enhancer could be activated using CRISPRa or other similar tools.

A combination of the above strategies: for example, combining the activation to increase transcription, along with knocking down the repressor, to further inhibit gene silencing, along with introducing a mutation that increases the stability of the zeta mRNA, to improve levels of zeta protein.

As discussed above and throughout, compositions and methods of the disclosure may be used for strategies focusing on increasing the production of alpha globin.

Compositions and methods of the disclosure may be used for decreasing production of gamma globin.

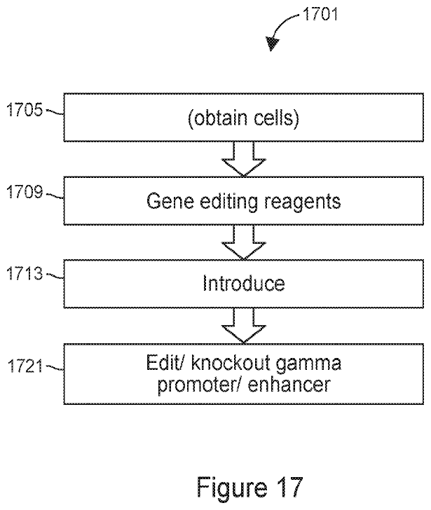

FIG. 17 diagrams a method 1701 for treating alpha thalassemia that can include the decrease of production of gamma globin. The method 1701 optionally includes obtaining 1705 fetal cells (e.g., via blood draw from a fetus). Gene editing reagents are prepared or obtained 1709 either ex vivo into the cells or in vivo into the fetus. In the cells (either ex vivo or in the fetus) the reagents either (i) increase production of alpha, zeta, or theta globin, or (ii) decrease production of gamma globin, or any combination thereof. Methods of increasing a globin are discussed above. The method 1701 may include decreasing or otherwise manipulating gamma globin to restore balance in the hemoglobin chains. Thus the gene editing reagents used in the method 1701 may include cognate sequences for a gamma-globin gene, its promoter, or its enhancer to downregulate expression of the gene or production of gamma globin.

Some approaches have sought to treat beta thalassemia by insertion of the genes into the globin locus or by increasing the production of gamma globin. For alpha thalassemia, the opposition approach is required. An approach to treating alpha thalassemia includes insertion of alpha-globin gene, or decreasing the production of gamma globin, or increasing the production of zeta globin.

Gamma globin is produced in large amounts by fetuses with ATM. In the fetal period, gamma would normally pair with alpha globin to make functional fetal hemoglobin. However, in the absence of adequate amounts of alpha globin, tetramers of gamma chains accumulate and RBCs containing these tetramers (Hb Bart's) are unable to deliver oxygen to fetal tissues. Since some zeta globin continues to be made, it is possible that decreasing the over-production of gamma could restore a normal ratio of zeta to gamma chains and enable improved oxygen delivery. Decreasing the production of gamma globin can therefore prevent the formation of abnormal Hb Bart's hemoglobin, which contributes to the severity of the disease. Gamma production could be decreased by targeting the known enhancers such as bcl11a or by other gene editing approaches.

Compositions and methods of the disclosure may be used for increasing the production of theta globin. Theta globin is 3′ of the alpha genes and has low transcription throughout development and postnatal life. Increasing production of theta globin may restore the balance of globins arising from the alpha and beta chains, with beneficial effects.

Compositions and methods of the disclosure may be used for treating alpha thalassemia. The compositions and methods use gene editing reagents such as one or more of a Cas endonuclease; a nucleic acid encoding the Cas endonuclease; a transcription activator-like effector nuclease (TALEN); a nucleic acid encoding the TALEN; a zinc-finger nuclease (ZFN); and a nucleic acid encoding the ZFN. The compositions and methods are useful ex vivo on cells (e.g., HSCs, RBCs, or precursors thereof), from the fetus to modify the cells or their progeny to obtain modified cells that are transplanted into the fetus. The compositions and methods are useful in vivo, by direct delivery of gene editing reagents to the fetus. The compositions and methods are useful to (i) increase production of alpha, zeta, or theta globin, or (ii) decrease production of gamma globin, or any combination thereof. The compositions and methods are useful to insert an alpha-globin gene; introduce a mutation into a repressor region in a zeta-globin gene; introduce a mutation into a 3′ sequence of a translated region of the zeta-globin gene; introducing a mutation into a gamma globin or its enhancer or promoter; or a combination thereof. Any of the gene editing strategies of the compositions and methods may beneficially be performed in conjunction with delivering to a fetus a therapeutic blood transfusion with blood that includes alpha globin. Embodiments include delivering a composition of the disclosure according to a clinical protocol that involves co-injecting the gene therapy/editing products with RBC transfusion to optimize the health of the fetus until the gene therapy approach becomes effective.

### EXAMPLES

### Example 1

### Engineered Erythroid Cells Model Alpha-Thalassemia Major (ATM) In Vitro

Developing strategies to treat ATM requires the availability of adequate cell models to test such strategies in vitro. HUDEP2 cells (Kurita et al. 2013) are immortalized human erythroid progenitor cells derived from umbilical cord blood of a healthy donor. They are a model for adult-type erythroid cells that express high levels of adult hemoglobin (beta- and alpha-globin) and little to no fetal and embryonic hemoglobin (gamma-, epsilon-, and zeta-globin). To model ATM, we generated HUDEP-2 cells that carry the most common ATM deletion (South-East-Asia deletion, ATM^{ΔSEA}) to use as a cell model to evaluate gene editing strategies and transgene expression.

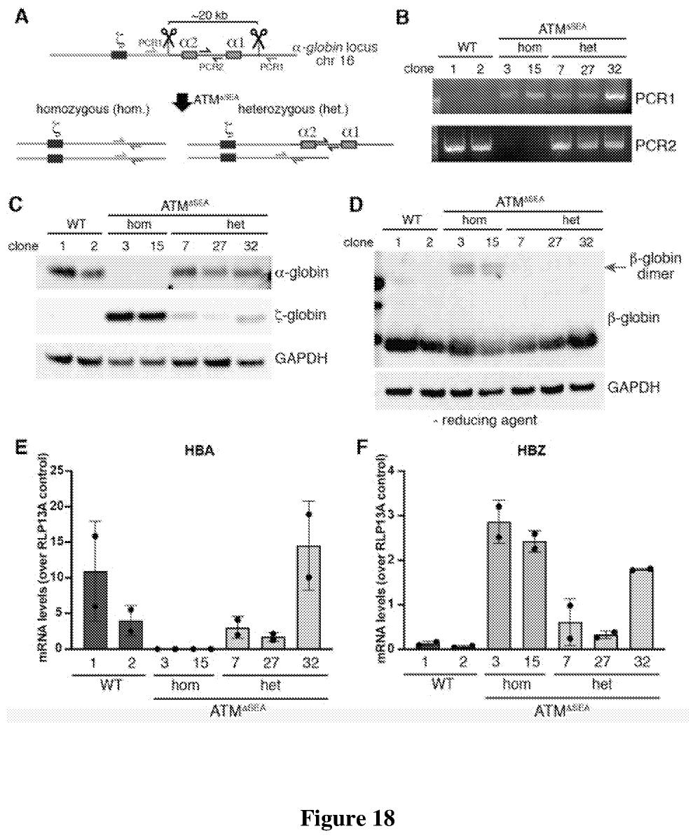

HUDEP2 cells were transfected with two Cas9-g RNA complexes targeting DNA sequences adjacent to the alpha-globin genes (FIG. 18A). Successful gene editing at both sites results in a deletion of about 20 kb encompassing the two alpha-globin genes and thus abolishing alpha-globin expression. Clonal populations of edited cells were established by sorting single cells into 96-well culture plates. Clones were screened by genomic PCR and clones that were either heterozygous or homozygous for the SEA deletion were selected (FIG. 18B). In addition we generated wild-type (i.e., WT) clonal populations to account for clone-to-clone variation. Clones were characterized for their globin expression levels by Western Blot (FIGS. 18C and 18D). Cells were lysed using RIPA buffer and whole protein extracts were size-separated by SDS-PAGE. Samples in FIG. 18C were denatured with reducing agent to break up disulfide bonds before gel electrophoresis. Samples in FIG. 18D were denatured without reducing agent to keep disulfide bonds intact. Furthermore, mRNA expression of globins was determined by quantitative real-time PCR (qPCR) using Taqman probes specific to either alpha- or zeta-globin (FIGS. 18E and 18F). Ct values for alpha- and zeta-globin were normalized to Ct values of a housekeeping gene (RPL13A).

We successfully edited and clonally expanded two homozygous and three heterozygous HUDEP2 clones carrying the ATM^{ΔSEA} deletion as determined by genomic PCR (FIG. 18B). The homozygous clones are missing all four alpha-globin genes, while the heterozygous clones maintain two copies of alpha-globin on one allele. We then confirmed these genotypes by measuring alpha-globin levels by Western Blot (FIG. 18C). As expected, alpha-globin levels were abolished in the homozygous ATM^{ΔSEA} clones and slightly reduced in the heterozygous clones. Interestingly, we found that zeta-globin expression was elevated in all ATM^{ΔSEA} clones, an observation that is coherent with observations made in patients with ATM^{ΔSEA} (Tang et al. 1992; Chung et al. 1984).

Furthermore, we were interested in the formation of beta-globin dimers that often form in red blood cells of patients with ATM due to the lack of alpha-globin chains. We found that homozygous ATM^{ΔSEA} cells indeed show the presence of beta-globin dimers (FIG. 18D). We confirmed our findings on the transcriptional level by determining alpha- and zeta-globin mRNA levels by qPCR (FIGS. 18E and 18F). Homozygous ATM^{ΔSEA} cells showed no expression of alpha-globin and significantly higher zeta-globin levels, while heterozygous clones had varying levels of alpha-globin expression and slightly elevated levels of zeta-globin expression.

The data establish that we successfully generated ATM^{ΔSEA} HUDEP2 cell models that we can utilize to study ATM. Furthermore, the cells mirror the phenotype that is observed in ATM patients, allowing us to confirm the value of ATM treatment strategies disclosed herein, such as genome editing approaches and transgene expression.

### Example 2

Alpha- and Zeta-Globin can be Expressed from a Transgene at High Levels

Genetic disorders that stem from loss of function mutations could potentially treated by replacing the missing gene with a transgene. In dividing cells, such as red blood cells, the transgene has to be permanently introduced into the host cell's genome to sustain long-term expression. Lentiviral vectors are a common medium to introduce transgenes and have successfully been used for the treatment of beta-thalassemia (Harrison 2019). For a therapeutic approach, alpha- or zeta-globin must be expressed at high levels from the transgene, and using a strong erythroid-specific promoter such as the beta-globin promoter is expected to boost expression levels in erythroid cells.

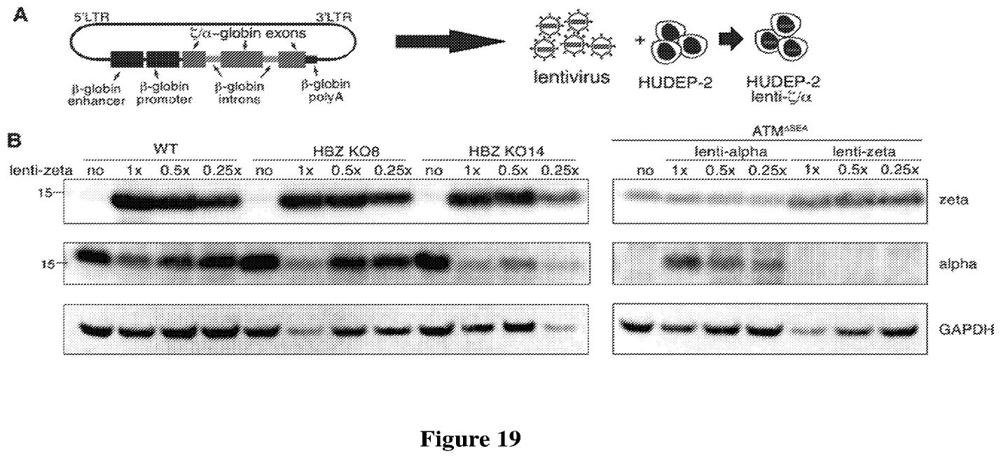

To assess the transgenic expression of alpha- and zeta-globin genes, we constructed lentiviral vectors containing alpha- or zeta-globin exons separated by beta-globin 3′UTR, intron 1 and 2 and the beta-globin polyA tail (FIG. 19A). Expression of the transgene is driven by the beta-globin promoter and by truncated sequences of hypersensitive sites 2 and 3 or the beta-globin enhancer (locus control region). Lentivirus was packaged in HEK293T cells and different amounts of viral supernatant were used to transduce HUDEP2 WT, zeta-globin knockout (HBZ KO, clone 8 and 14) and ATM^{ΔSEA} cells (FIG. 19A). HUDEP2 cells were cultured for several days after transduction and then harvested for analysis. Cells were lysed using RIPA buffer and whole protein extracts were size-separated by SDS-PAGE and transferred to a nitrocellulose membrane. Membranes were probed with antibodies against alpha-globin, zeta-globin and GAPDH as a loading control (FIG. 19B).

Beta-globin regulatory sequences, introns and UTRs were maintained in the lentiviral expression plasmids (FIG. 19A). Only the exons of beta-globin were swapped for exons of either alpha- or zeta-globin. High, dose-dependent expression of zeta-globin was observed after transduction of HUDEP2 WT, HBZ KO and ATM^{ΔSEA} cells with lenti zeta-globin. Similarly high expression was observed in HUDEP2 ATM^{ΔSEA} cells after transduction with lenti alpha-globin (FIG. 19B).

Both alpha- and zeta-globin were expressed at high levels from a transgene that was introduced by lentiviral transduction into erythroid progenitor cells and expression levels correlated with viral titer used for transduction. Furthermore, the beta-globin promoter and enhancer represent strong regulatory elements to drive the expression of alpha- and zeta-globin in adult-type erythroid cells. The data establish that high levels of expression of alpha globin and zeta globin can be achieved ex vivo from transgenes in cultured cells.

### Example 3

Gene Editing Strategies to Knock Alpha-Globin into the Beta-Globin Locus

ATM severity manifests in the lack of alpha-globin chains available to form functional adult hemoglobin (α₂β₂), thus causing severe anemia and hypoxia. Besides the lack of alpha-globin chains, the excess amount of beta-globin chains also causes major issues as unpaired beta-globin chains form toxic precipitates. Thus, reducing the amount of beta-globin at the same time as elevating the expression of alpha-globin is expected to be a viable strategy for the treatment of ATM. Maintaining the correct balance of hemoglobin chains is crucial and, hence, using endogenous gene regulatory elements to regulate expression of alpha-globin could be advantageous. Thus, the aim was to knock alpha-globin into the endogenous beta-globin locus in a heterozygous manner by homologous recombination. Successful knock-in leads to the replacement of beta-globin with alpha-globin. As this process is fairly inefficient in hematopoietic stem cells, only about 50% of alleles will be successfully targeted, leaving 50% of beta-globin alleles intact. Targeting the nucleases used to engineer homologous recombination of DNA to intronic sequences of beta-globin will ensure that cells that fail to undergo homologous recombination will have indels in non-translated regions of beta-globin, thereby ensuring the sustained expression of functional beta-globin.

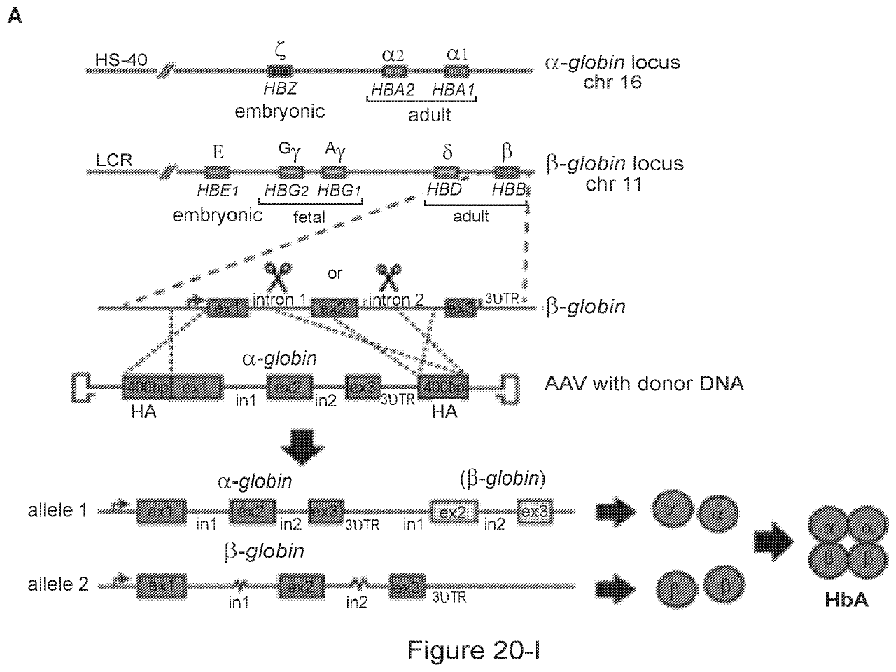

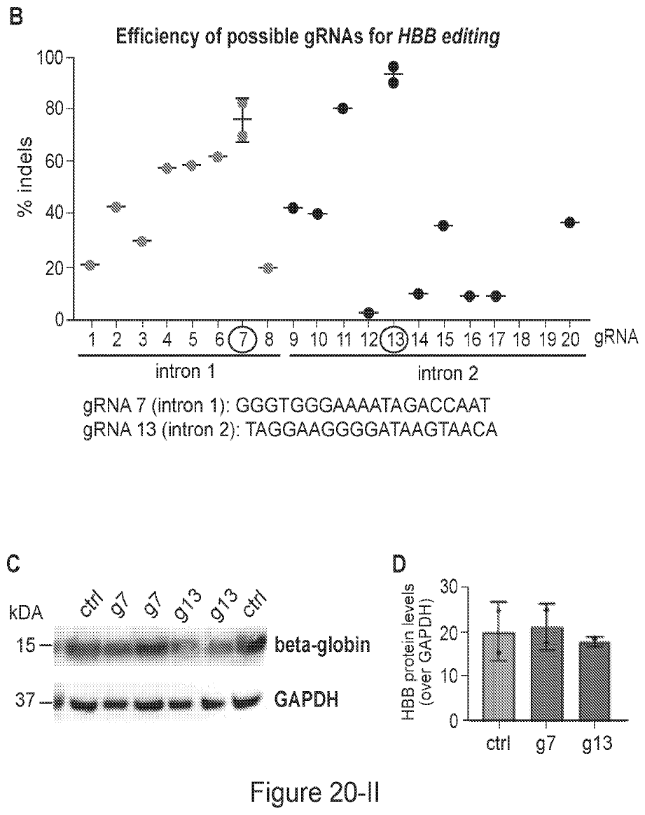

The knock-in strategy was designed to make a nuclease cut in either intron 1 or intron 2 of beta-globin and donor DNA within an AAV vector (FIG. 20A). The donor DNA has 400 bp of homology to the translation start site of beta globin and 400 bp of homology downstream of the nuclease cut site. The donor DNA contains the alpha-globin gene sequence including exons and introns and the alpha-globin 3′UTR. 20 different gRNAs (8 in intron 1 and 12 in intron 2) were tested for their editing efficiency in HUDEP2 WT cells. Guide RNAs were complexed with high-fidelity Cas9 nuclease and HUDEP2 were nucleofected with ribonucleoprotein. After 5 days, cells were harvested and genomic DNA and protein was extracted. Editing efficiencies were determined by running a PCR across the edited locus, Sanger sequencing the PCR products, and analyzing insertion and deletion (indel) frequencies by ICE (Inference of CRISPR Edits; Synthego) (FIG. 20B). Wild-type (WT) cells and cells edited with gRNA 7 and 13 were lysed using RIPA buffer and whole protein extracts were size-separated by SDS-PAGE and transferred to a nitrocellulose membrane. Membranes were probed with antibodies against beta-globin and GAPDH as a loading control (FIG. 20C). Protein amounts from Western Blot were quantified using the LiCor software and beta-globin expression levels were normalized to GAPDH levels (FIG. 20D).

Out of 8 gRNAs tested targeting intron 1 of beta-globin, one gRNA (gRNA 7) showed very high levels of indels. For intron 2, two gRNAs showed editing levels above 80% (gRNA 11 and 13) with gRNA13 showing the highest editing levels (FIG. 20B). To ensure beta-globin expression is unaffected by the indels produced, Western Blot analysis was performed. Neither of the two high-indel producing gRNAs tested (gRNA 7 and 13) significantly reduced the expression of beta-globin in HUDEP2 cells.

Two gRNAs were identified that were suitable to introduce alpha-globin into the beta-globin locus by AAV-mediated homologous recombination. The indels produced by these gRNAs did not affect the expression of beta-globin in erythroid progenitor cells and thus represent good candidates for therapeutic gene editing in hematopoietic stem cells.

### Example 4

Targeting Genetic Elements within the Alpha-Globin Cluster Derepresses Zeta-Globin

Embryonic zeta-globin could compensate for the lack of alpha-globin in ATM patients. Normally, zeta-globin in silenced after the first trimester, when alpha globin begins to be expressed. As many ATM patients have intact embryonic zeta-globin genes, reactivating the expression of zeta-globin in adult erythroid cells could provide a therapeutic strategy for the treatment of ATM. Targeting DNA sequences that are required for zeta-globin silencing using a nuclease is expected to disrupt the binding site of respective repressors and prohibit silencing of zeta-globin. Little is known about how zeta-globin is silenced, thus investigating these mechanisms is expected to reveal targetable genetic elements for gene therapy.

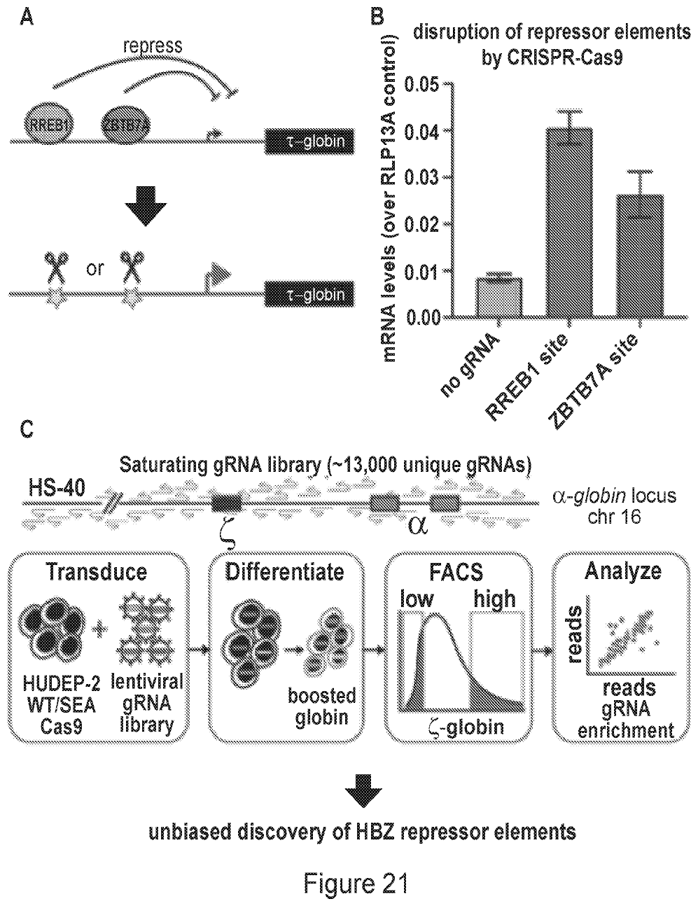

Guide RNAs were designed to target the binding sites of RREB1 and ZBTB7A in the zeta-globin promoter (FIG. 21A). HUDEP2 cells were nucleofected with Cas9 and respective gRNAs and harvested 5 days post-nucleofection. Total mRNA was extracted and cDNA was synthesized. Expression levels were quantified by real-time qPCR using Taqman probes specific to zeta-globin (FIG. 21B). Ct values for zeta-globin were normalized to Ct values of a housekeeping gene (RPL13A). A library of about 13,000 unique gRNAs tiling the entire alpha-globin locus was cloned into a lentiviral expression plasmid. Lentivirus was produced in HEK293T cells and lentiviral supernatant was used to transduce HUDEP2 cells stably expressing Cas9 and low multiplicity of infection (MOI 0.2). Infected cells were FACS-sorted (for the expression of the transgene) and differentiated for 4 days. Cells were intracellularly stained with a zeta-globin antibody and FACS-sorted into high-expressing (top 10%) and low-expressing (low 10%) bins. Genomic DNA was extracted from those populations, the lentiviral cassette amplified by PCR and the products sequenced by Illumina next-generation sequencing.

The transcription factors RREB1 and ZBTB7A have been reported to repress zeta-globin gene expression (Masuda et al. 2016; Chen et al. 2010). The zeta-globin promoter contains binding motifs for these two factors that were targeted with a Cas9 nuclease with the aim of disrupting binding in HUDEP2 cells (FIG. 21A). Edited cells showed elevated levels of zeta-globin mRNA expression compared to wild-type (WT) HUDEP2 cells, as measured by real-time qPCR (FIG. 21B). To detect other regulatory elements that are essential for zeta-globin silencing, an unbiased approach was taken by performing a CRISPR tiled screen across the entire alpha-globin locus (FIG. 21C).

The results establish that targeting specific repressor binding sites is an effective way to reactivate the expression of zeta-globin. In order to find the most effective site to target, an unbiased CRISPR screen was performed. The results of this screen are expected to uncover genetic elements targetable with a nuclease to reactivate the expression of zeta-globin in erythroid cells.

### REFERENCES

Chen, R.-L., Chou, Y.-C., Lan, Y.-J., Huang, T.-S. and Shen, C.-K. J. 2010. Developmental silencing of human zeta-globin gene expression is mediated by the transcriptional repressor RREB1. The Journal of Biological Chemistry 285(14), pp. 10189-10197.
Chung, S. W., Wong, S. C., Clarke, B. J., Patterson, M., Walker, W. H. and Chui, D. H. 1984. Human embryonic zeta-globin chains in adult patients with alpha-thalassemias. Proceedings of the National Academy of Sciences of the United States of America 81(19), pp. 6188-6191.
Harrison, C. 2019. First gene therapy for β-thalassemia approved. Nature Biotechnology.
Kurita, R., Suda, N., Sudo, K., Miharada, K., Hiroyama, T., Miyoshi, H., Tani, K. and Nakamura, Y. 2013. Establishment of immortalized human erythroid progenitor cell lines able to produce enucleated red blood cells. Plos One 8(3), p. e59890.
Masuda, T., Wang, X., Maeda, M., Canver, M. C., Sher, F., Funnell, A. P. W., Fisher, C., Suciu, M., Martyn, G. E., Norton, L. J., Zhu, C., Kurita, R., Nakamura, Y., Xu, J., Higgs, D. R., Crossley, M., Bauer, D. E., Orkin, S. H., Kharchenko, P. V. and Maeda, T. 2016. Transcription factors LRF and BCL11A independently repress expression of fetal hemoglobin. Science 351(6270), pp. 285-289.
Tang, W., Luo, H. Y., Albitar, M., Patterson, M., Eng, B., Waye, J. S., Liebhaber, S. A., Higgs, D. R. and Chui, D. H. 1992. Human embryonic zeta-globin chain expression in deletional alpha-thalassemias. Blood 80(2), pp. 517-522

Each of the references cited herein is hereby incorporated by reference in its entirety or in relevant part, as would be apparent from the context of the citation.

It is to be understood that while the claimed subject matter has been described in conjunction with the detailed description thereof, the foregoing description is intended to illustrate and not limit the scope of that claimed subject matter, which is defined by the scope of the appended claims. Other aspects, advantages, and modifications are within the scope of the following claims.

What is claimed is:

1. A method of treating alpha thalassemia, the method comprising:
obtaining a sample comprising HSCs from a fetus or patient no older than one year of age by administering a composition comprising:
(a) a globin gene, wherein the globin gene is an alpha-globin gene, a zeta-globin gene, a zeta- or alpha-globin gene associated with at least one beta-globin regulatory region, a zeta- or alpha-globin gene associated with at least one beta-globin intron, or a zeta- or alpha-globin gene associated with at least on beta-globin regulatory region and at least one beta-globin intron; and
(b) gene editing reagents that, when the composition is introduced directly into the fetus or patient, or into HSCs derived from the fetus or patient, insert the globin gene into genomic material to introduce the globin gene into the HSCs or progeny of the HSCs.

2. The method of claim 1, further comprising surgically accessing the fetus in the pregnant woman and introducing the HSC into fetal circulation by injection.

3. The method of claim 1 wherein the gene editing reagents, when introduced into a fetus or patient no older than one year of age, or into cells obtained therefrom, introduce a change into a sequence within a globin gene within genomic material of the fetus, patient, cells, or progeny thereof, wherein the change activates or derepresses the globin gene to modify the expression of the zeta-globin gene within the cells.

4. The method of claim 3, wherein the gene editing reagents are introduced into fetal circulation, further wherein the modified globin gene is a modified zeta-globin gene that is resistant to gene silencing and is expressed persistently into at least a second trimester.

5. The method of claim 3, wherein the globin gene is inserted into the genomic material and expressed in the fetus or patient no older than one year of age.

6. The method of claim 5, further comprising introducing the cells or progeny thereof into fetal circulation by injection into an umbilical cord, placenta, liver, or heart of the fetus.

7. The method of claim 6, wherein the gene editing reagents comprise at least one guide RNA and at least one Cas endonuclease or a nucleic acid encoding the Cas endonuclease.

8. The method of claim 1, wherein the globin gene is an alpha-globin gene and the at least one guide RNA targets delivery of the alpha-globin gene to a predetermined locus in the genomic material, wherein the locus is selected from an alpha-globin gene cluster in chromosome 16, an intronic region of beta globin in chromosome 11, and a genomic safe harbor.

9. The method of claim 1, wherein the globin gene is included as DNA and the gene editing reagents include at least one mRNA that, when introduced into the cells or fetus, is translated into a gene editing nuclease.

10. The method of claim 7, wherein the gene editing reagents comprise at least a first Cas9 ribonucleoprotein (RNP) that includes a first guide RNA (g RNA) that binds the RNP to a locus within a globin gene cluster in the genomic material; and introduces the globin gene into the locus within the globin gene cluster.

11. A method for treating alpha thalassemia, the method comprising introducing into a fetal cell, wherein the fetal cell comprises an HSC, RBC, or precursor thereof, or into circulation of a fetus, gene editing reagents that (i) increase production of alpha, zeta, or theta globin, (ii) decrease production of gamma globin, or (iii) decrease production of gamma globin and increase production of zeta globin.

12. The method of claim 11, wherein the decreased production of gamma globin is due to a knockout mutation of gamma-globin.

13. The method of claim 11, wherein the zeta-globin gene is introduced into the fetal cell by insertion into the gamma-globin gene, thereby decreasing production of gamma globin and increasing production of zeta globin.

14. The method of claim 11, wherein the gene editing reagents include at least one composition selected from the group consisting of a Cas endonuclease and a guide RNA, a nucleic acid encoding the Cas endonuclease and a nucleic acid encoding a guide RNA, a transcription activator-like effector nuclease (TALEN), a nucleic acid encoding the TALEN, a zinc-finger nuclease (ZFN), and a nucleic acid encoding the ZFN.

15. The method of claim 11, wherein the gene editing reagents:
(a) introduce a mutation into a repressor region in a zeta-globin gene; and
(b) introduce a mutation into a 3′ sequence of a translated region of a zeta-globin gene.

16. The method of claim 15, further comprising administering gene editing reagents to the fetus to inhibit gene silencing of a zeta-globin gene and to increase persistence of zeta globin into at least a second trimester in the fetus, wherein the gene editing reagents include a Cas endonuclease gene and a DNA-sense guide RNA for introducing a mutation into a repressor region in the zeta-globin gene or for introducing a mutation into a 3′ sequence of a transcribed region of the zeta-globin gene.

17. The method of claim 11, wherein the fetal cell is a hematopoietic stem cell (HSC), wherein the gene editing reagents introduce an activating mutation in the zeta-globin gene of the HSC ex vivo, and wherein the mutated HSC or progeny thereof is delivered to the fetal circulation by injection into the fetus, umbilical cord, or placenta.

18. A composition for treatment of alpha thalassemia in a fetus or a patient no older than one year of age, or a cell thereof, the composition comprising:
(a) a globin gene, wherein the globin gene is an alpha-globin gene, a zeta-globin gene, a zeta- or alpha-globin gene associated with at least one beta-globin regulatory region, a zeta- or alpha-globin gene associated with at least one beta-globin intron, or a zeta- or alpha-globin gene associated with at least on beta-globin regulatory region and at least one beta-globin intron; and
(b) gene editing reagents that, when the composition is introduced directly into the fetus or patient, or into HSCs derived from the fetus or patient, insert the globin gene into genomic material.

19. The composition of claim 18, wherein the globin gene is an alpha-globin gene and the gene editing reagents comprise a first Cas9 ribonucleoprotein (RNP) that includes a first guide RNA (gRNA) and a second Cas9 RNP, wherein the first Cas9 RNP and the second Cas9 RNP bind to a locus within an alpha-globin gene cluster in chromosome 16 of the genomic material, and introduce the alpha-globin gene into the locus within the alpha-globin gene cluster.

20. The composition of claim 18, wherein the gene editing reagents are targeted to a predetermined locus in the genomic material, wherein the locus is selected from:
an alpha-globin gene cluster in chromosome 16;
an intronic region of the beta globin gene in chromosome 11, and
an AAVS1, CCR5, CLYBL or hROSA26 genomic safe harbor.

21. The composition of claim 18, wherein the gene editing reagents comprise an mRNA that is translated into a gene editing nuclease selected from the group consisting of a Cas endonuclease, a pair of transcription activator-like effector nucleases (TALEN), and a pair of zinc-finger nucleases (ZFN).

22. The composition of claim 18, wherein the gene editing reagents comprise at least one guide RNA that targets the globin gene and a Cas endonuclease or nucleic acid encoding a Cas endonuclease, wherein the gene editing reagents, when introduced into a fetus, a patient no older than one year of age, or into cells obtained therefrom, introduce a change into a sequence within a globin gene within genomic material of the fetus, patient, cells, or progeny thereof, wherein the change activates or derepresses the globin gene.

23. The composition of claim 18, wherein the gene editing reagents introduce a mutation into a ZBTB7A binding site, a RREB1 binding site, or a NF-kB binding site in a repressor region in a 3′ end of the zeta-globin gene.

24. The composition of claim 18, wherein the gene editing reagents include CRISPR, TALENS, or ZFNs, and are included in the composition in a form that includes DNA, mRNA, or protein.
